(19) United States
(12) Patent Application Publication
(10) Pub. No.: US 2024/0400471 A1
(43) Pub. Date: Dec. 5, 2024

(54) LABELING METHOD, OXIDANT FOR LABELING, RUTHENIUM COMPLEX, CATALYST, LABELING COMPOUND, AND COMPOUND

(71) Applicant: KYUSHU UNIVERSITY, NATIONAL UNIVERSITY CORPORATION, Fukuoka (JP)

(72) Inventors: Tatsuya UCHIDA, Fukuoka-shi, Fukuoka (JP); Daiki DOIUCHI, Fukuoka-shi, Fukuoka (JP); Tatsuya NAKAMURA, Fukuoka-shi, Fukuoka (JP); Nanako SHIMODA, Fukuoka-shi, Fukuoka (JP)

(73) Assignee: KYUSHU UNIVERSITY, NATIONAL UNIVERSITY CORPORATION, Fukuoka (JP)

(21) Appl. No.: 18/258,739

(22) PCT Filed: Dec. 22, 2021
(86) PCT No.: PCT/JP2021/047658

§ 371 Date: Jul. 25, 2023

(30) Foreign Application Priority Data
Dec. 23, 2020 (JP) 2020-213969

(51) Int. Cl. C07B 59/00; C07F 15/00

(52) U.S. Cl. CPC C07B 59/002; C07F 15/0053; C07B 2200/05

(57) ABSTRACT

Provided is a labeling method having a step of labeling a substrate having a carbon-hydrogen bond with an oxygen isotope by using a catalyst and an oxidant produced from a hypervalent iodine compound having an ester structure and labeled water labeled with at least one oxygen isotope selected from the group consisting of ¹⁷O and ¹⁸O. Also provided is an oxidant for labeling that is produced from a hypervalent iodine compound having an ester structure and labeled water labeled with at least one oxygen isotope selected from the group consisting of ¹⁷O and ¹⁸O and labels a substrate having a carbon-hydrogen bond with an oxygen isotope in the co-presence of a catalyst.

### TECHNICAL FIELD

The present disclosure relates to a labeling method using an oxygen isotope, an oxidant for labeling using an oxygen isotope, a ruthenium complex, a catalyst, a labeled compound using an oxygen isotope, and a novel compound.

### BACKGROUND ART

Attempts have been made to administer a labeled compound, in which a portion of an element constituting a chemical substance is labeled with an isotope, into a living body and obtain knowledge on the metabolic pathway in the living body and the distribution of the substance in the living body. Many biologically active compounds include an oxygen functional group such as a hydroxy group or a carbonyl group. Along with the advancement in the measuring apparatuses and measurement methods in recent years, it is expected that ¹⁷O and ¹⁸O, which are stable isotopes of oxygen atoms, are utilized for in vivo imaging. Regarding a method for introducing ¹⁷O and ¹⁸O into an organic oxygen compound, a technology of using a hydration reaction into an alkene or a carbonyl group, or a technology of converting the coordination water of a metal complex into a metal oxo species by using electrolysis or an oxidant, are known. Furthermore, in Patent Literature 1, it has been proposed to use an oxygen isotope-labeled carboxylic acid salt compound as a supply source of an oxygen isotope.

### CITATION LIST

### Patent Literature

Patent Literature 1: Japanese Unexamined Patent Publication No. 2020-37545

### SUMMARY OF INVENTION

### Technical Problem

However, in conventional methods of using oxygen isotope-labeled water, it is necessary to carry out the methods by using a large excess of oxygen isotope-labeled water. From the viewpoint of resource conservation of rare oxygen isotope-labeled water, it is desirable to improve such conventional methods. Furthermore, in the method of Patent Literature 1, it is necessary to synthesize an oxygen isotope-labeled carboxylic acid salt compound in order to label a substrate. For this reason, there is a demand for a technology that enables convenient labeling by means of an oxygen isotope without consuming a large amount of oxygen isotope-labeled water.

Thus, the present disclosure provides a labeling method using an oxygen isotope, by which a labeled compound can be obtained in high yield without using an excess of oxygen isotope-labeled water. Furthermore, an oxidant for labeling, a ruthenium complex, and a catalyst, which can be suitably used in such a labeling method, are provided. Furthermore, a labeled compound labeled by means of an oxygen isotope is provided. Furthermore, a novel compound useful as a reagent is provided.

### Solution to Problem

According to an aspect of the present disclosure, there is provided a labeling method having a step of labeling a substrate having a carbon-hydrogen bond with an oxygen isotope by using a catalyst and an oxidant produced from a hypervalent iodine compound having an ester structure and labeled water labeled with at least one oxygen isotope selected from the group consisting of ¹⁷O and ¹⁸O.

In the step of the labeling method, a carbon-hydrogen bond of the substrate is oxidized with high regioselectivity by using the oxidant and the catalyst. By using the oxidant produced from the hypervalent iodine compound having an ester structure and the labeled water, the substrate can be labeled with isotopic oxygen. For this reason, a labeled compound can be obtained in high yield without using a large amount of oxygen isotope-labeled water.

The catalyst may include a ruthenium complex. The catalyst may include at least one ruthenium complex selected from the group consisting of the following General Formulas (1), (2), and (3).

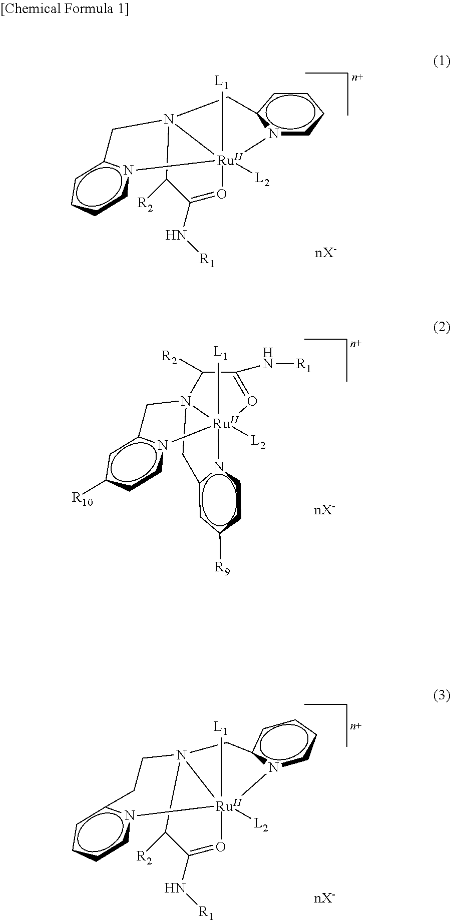

In the General Formulas (1), (2), and (3), R₁ represents a hydrogen atom, a phenyl group, or a monovalent group having at least one hydrogen atom of a phenyl group substituted with an alkyl group, a hydroxy group, a phenyl group, a halogen atom, or an alkoxy group; R₂ represents a hydrogen atom, a phenyl group, or an alkyl group; L₁ represents a halogen atom or a water molecule; L₂ represents triphenylphosphine, pyridine, imidazole, or dimethyl sulfoxide; X represents a halogen atom; and n represents 1 or 2. In the General Formula (2), R₉ and R₁₀ each independently represent a hydrogen atom, a halogen atom, or an alkyl group.

The catalyst including a ruthenium complex has excellent activity for an oxidation reaction of a carbon-hydrogen bond and also has excellent regioselectivity. Therefore, a compound labeled with an intended oxygen isotope can be obtained in high yield from various substrates having a carbon-hydrogen bond.

The hypervalent iodine compound used in the labeling method may include a compound represented by the following General Formula (4)

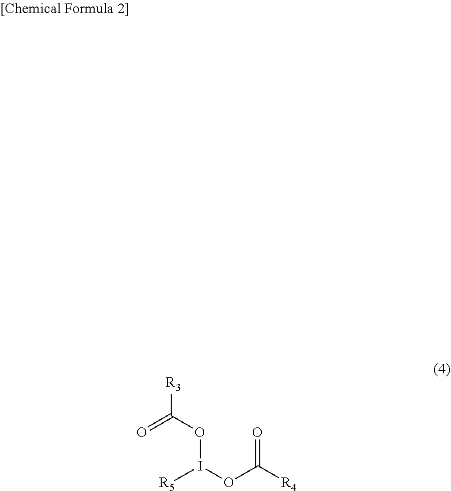

In the General Formula (4), R₃ and R₄ each independently represent a hydrogen atom, an alkyl group, or a monovalent group having an aromatic ring; and R₅ represents a monovalent group having an aromatic ring.

In the step, the compound represented by the General Formula (4) can activate labeled water and promote a reaction between a substrate and labeled water. As a result, an intended oxygen isotope-labeled compound can be obtained in high yield.

In the step, a hydroxy compound or an oxo compound labeled with an oxygen isotope may be obtained by oxidizing the substrate. Such a hydroxy compound or oxo compound can be used in various use applications.

In the step, a hexose may be labeled by substituting an oxygen atom of a hexose included in a substrate with an oxygen isotope. The hexose labeled with an oxygen isotope, which is obtained in this way, can be utilized as, for example, a molecular probe labeled with an oxygen isotope in in-vivo imaging such as observation of cellular tissue.

According to an aspect of the present disclosure, there is provided an oxidant for labeling that is produced from a hypervalent iodine compound having an ester structure and labeled water labeled with at least one oxygen isotope selected from the group consisting of ¹⁷O and ¹⁸O, and labels a substrate having a carbon-hydrogen bond with an oxygen isotope in the co-presence of a catalyst. This oxidant for labeling can oxidize a carbon-hydrogen bond of the substrate with high regioselectivity in the co-presence of the catalyst and label the substrate with isotopic oxygen. For this reason, a labeled compound can be obtained in high yield without using a large amount of oxygen isotope-labeled water.

The hypervalent iodine compound used in the labeling method may include a compound represented by the following General Formula (4). At least one of oxygen atoms in the following General Formula (4) may be ¹⁷O or ¹⁸O.

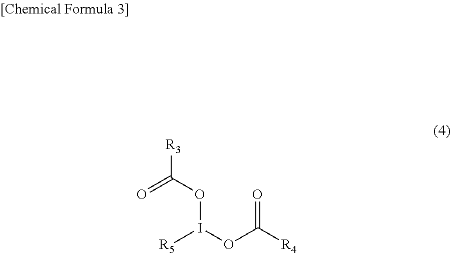

In the General Formula (4), R₃ and R₄ each independently represent a hydrogen atom, an alkyl group, or a monovalent group having an aromatic ring; and R₅ represents a monovalent group having an aromatic ring.

By using a hypervalent iodine compound including the compound represented by the General Formula (4), an intended oxygen isotope-labeled compound can be obtained in high yield by stably reacting the substrate with the oxidant.

According to an aspect of the present disclosure, there is provided a ruthenium complex represented by the following General Formula (2) or (3).

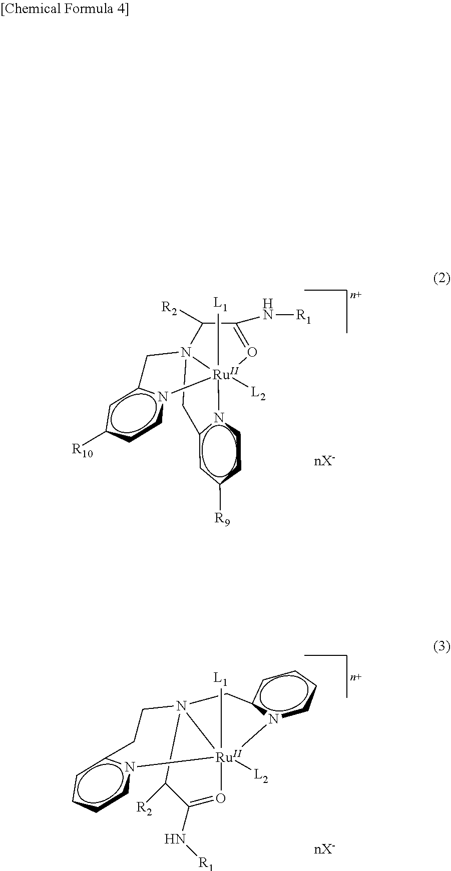

In the General Formulas (2) and (3), R₁ represents a hydrogen atom, a phenyl group, or a monovalent group having at least one hydrogen atom of a phenyl group substituted with an alkyl group, a hydroxy group, a phenyl group, a halogen atom, or an alkoxy group; R₂ represents a hydrogen atom, a phenyl group, or an alkyl group; L₁ represents a halogen atom or a water molecule; L₂ represents triphenylphosphine, pyridine, imidazole, or dimethyl sulfoxide; X represents a halogen atom; and n represents 1 or 2. In the General Formula (2), R₉ and R₁₀ each independently represent a hydrogen atom, a halogen atom, or an alkyl group.

The ruthenium complex has high activity as a catalyst in a reaction of oxidizing a carbon-hydrogen bond. Such a ruthenium complex can be used as a catalyst in various use applications. For example, a substrate having a carbon-hydrogen bond can be oxidized with high regioselectivity. For this reason, the ruthenium complex can oxidize a carbon-hydrogen bond of a substrate with high regioselectivity as an oxidation catalyst in the co-presence of an oxidant produced from a hypervalent iodine compound having an ester structure and labeled water labeled with an oxygen isotope, and can label the substrate with ¹⁷O or ¹⁸O. That is, the ruthenium complex is useful as a catalyst for oxygen isotope labeling. However, the use application of the ruthenium complex is not limited to the above-mentioned applications. For example, the ruthenium complex may be an oxidation catalyst that oxidizes a substrate without labeling.

According to an aspect of the present disclosure, there is provided a catalyst including at least one selected from the group consisting of the ruthenium complex represented by the General Formula (2) and the ruthenium complex represented by the General Formula (3). Such a catalyst exhibits high activity in a reaction of oxidizing a carbon-hydrogen bond. The catalyst may be an oxidation catalyst that oxidizes a substrate having a carbon-hydrogen bond or may be an oxidation catalyst that hydroxylates a substrate.

According to an aspect of the present disclosure, there is provided a labeled compound represented by the following Formula (5), (6), or (7), the labeled compound being labeled with at least one oxygen isotope selected from the group consisting of ¹⁷O and ¹⁸O. Such a labeled compound can be used in various use applications. For example, the labeled compound can be utilized in in-vivo image as a molecular probe.

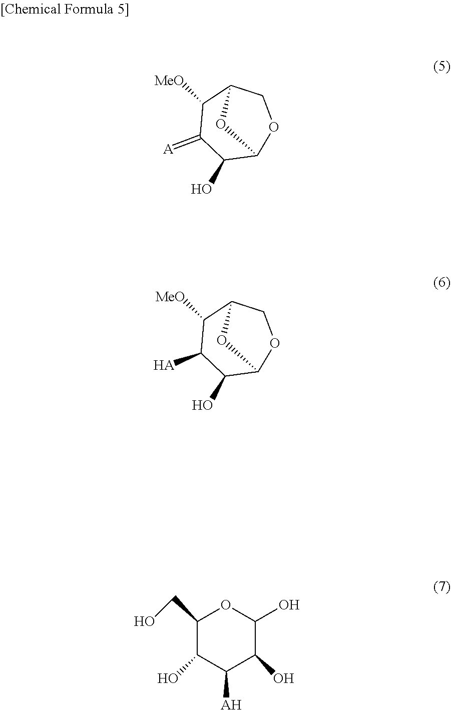

In the Formulas (5), (6), and (7), A represents ¹⁷O or ¹⁸O. The labeled compound is labeled with ¹⁷O or ¹⁸O. Such a labeled compound can be used in various use applications. For example, the labeled compound can be utilized in in-vivo image as a molecular probe.

According to an aspect of the present disclosure, there is provided a compound (novel compound) represented by the following Formula (8). Incidentally, Me in Formula (8) represents a methyl group.

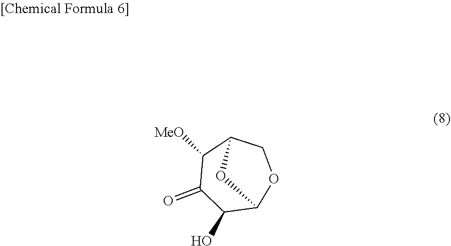 [SMILES: CO[C@H]1C(=O)[C@H](O)[C@@H]2OC[C@H]1O2]

The compound can be conveniently labeled with an oxygen isotope. For example, the compound is useful as an intermediate for obtaining mannose labeled with isotopic oxygen. This novel compound can be used as, for example, an intermediate for producing mannose labeled with isotopic oxygen from mannose.

### Advantageous Effects of Invention

According to the present disclosure, a labeling method using an oxygen isotope, by which a labeled compound can be obtained in high yield without using an excess of oxygen isotope-labeled water, can be provided. Furthermore, an oxidant for labeling, a ruthenium complex, and a catalyst, all of which can be suitably used for such a labeling method, can be provided. Furthermore, a labeled compound labeled by means of an oxygen isotope can be provided. Furthermore, a novel compound useful as a reagent can be provided.

### BRIEF DESCRIPTION OF DRAWINGS

FIG. 1 is a diagram showing an example of a production mechanism for alcohol when ruthenium complex is used as a catalyst.

FIG. 2 is a diagram showing ¹H-NMR measurement results of a ruthenium complex of Formula [III] (trans-form), a ruthenium complex of Formula [IV] (cis-form), and a mixture thereof.

FIG. 3 is a diagram showing the results of a single crystal structure analysis of the ruthenium complex of Formula [III].

FIG. 4 is a diagram showing the results of a single crystal structure analysis of the ruthenium complex of Formula [III] viewed from an angle different from that of FIG. 3.

FIG. 5 is a diagram showing the results of a single crystal structure analysis of the ruthenium complex of Formula [IV].

FIG. 6 is a diagram showing the results of a single crystal structure analysis of the ruthenium complex of Formula [IV] viewed from an angle different from that of FIG. 5.

FIG. 7 is a diagram showing the results of a single crystal structure analysis of a ruthenium complex of Formula (V).

FIG. 8 is a diagram showing the results of a single crystal structure analysis of the ruthenium complex of Formula (V) viewed from an angle different from that of FIG. 7.

FIG. 9 shows the results of time-of-flight mass spectrometry of adamantan-1-ol labeled with an oxygen isotope in Example 2-1.

FIG. 10 shows the results of time-of-flight mass spectrometry of 7-hydroxy-3,7-dimethyloctyl acetate labeled with an oxygen isotope in Example 2-2.

FIG. 11 shows the results of time-of-flight mass spectrometry of 7-hydroxy-3,7-dimethyloctyl acetate labeled with an oxygen isotope in Example 2-3.

FIG. 12 shows the results of time-of-flight mass spectrometry of 4-hydroxy-4-methylpentyl benzoate labeled with an oxygen isotope in Example 2-4.

FIG. 13 shows the results of time-of-flight mass spectrometry of (1R,2R,4R,5R)-4-hydroxy-2-methoxy-6,8-dioxabicyclo[3.2.1]octan-3-one labeled with oxygen-18 isotope in Example 3-1.

FIG. 14 shows the results of ¹H-NMR analysis of (1R,2R,4R,5R)-4-hydroxy-2-methoxy-6,8-dioxabicyclo[3.2.1]octan-3-one labeled with oxygen-18 isotope in Example 3-1.

FIG. 15 shows the results of two-dimensional NMR analysis of (1R,2R,4R,5R)-4-hydroxy-2-methoxy-6,8-dioxabicyclo[3.2.1]octan-3-one labeled with oxygen-18 isotope in Example 3-1.

FIG. 16 shows the results of ¹³C-NMR analysis based on BCM of (1R,2R,4R,5R)-4-hydroxy-2-methoxy-6,8-dioxabicyclo[3.2.1]octan-3-one labeled with oxygen-18 isotope in Example 3-1.

FIG. 17 shows the results of ¹³C-NMR analysis based on a DEPT method of (1R,2R,4R,5R)-4-hydroxy-2-methoxy-6,8-dioxabicyclo[3.2.1]octan-3-one labeled with oxygen-18 isotope in Example 3-1.

FIG. 18 shows the results of time-of-flight mass spectrometry of 1,6-anhydro-4-O-methyl-β-D-mannopyranose labeled with oxygen-18 isotope in Example 3-2.

FIG. 19 shows the results of ¹H-NMR analysis of 1,6-anhydro-4-O)-methyl-β-D-mannopyranose labeled with oxygen-18 isotope in Example 3-2.

FIG. 20 is a diagram showing the substituents of ruthenium complexes used in Examples 4-2 to 4-8 and the amine compounds used when obtaining the ruthenium complexes.

FIG. 21 shows the results of ¹H-NMR analysis of ligand 2 obtained in Example 5-1.

FIG. 22 shows the results of ¹³C-NMR analysis of the ligand 2 obtained in Example 5-1.

FIG. 23 shows the results of time-of-flight mass spectrometry of the ligand 2 obtained in Example 5-1.

FIG. 24 shows the results of ¹H-NMR analysis of a ruthenium complex obtained in Example 5-1.

FIG. 25 shows the results of time-of-flight mass spectrometry of the ruthenium complex obtained in Example 5-1.

FIG. 26 shows the results of ¹H-NMR analysis of ligand 3 obtained in Example 5-2.

FIG. 27 shows the results of ¹³C-NMR analysis of the ligand 3 obtained in Example 5-2.

FIG. 28 shows the results of time-of-flight mass spectrometry of the ligand 3 obtained in Example 5-2.

FIG. 29 shows the results of ¹H-NMR analysis of a ruthenium complex obtained in Example 5-2.

FIG. 30 shows the results of time-of-flight mass spectrometry of the ruthenium complex obtained in Example 5-2.

FIG. 31 is a graph showing changes over time in the natural logarithm of the relative ratio between the concentration [S] of a substrate as determined from the conversion efficiency at each reaction time and the initial concentration [S₀] of the substrate.

### DESCRIPTION OF EMBODIMENTS

Embodiments of the present disclosure will be described below. However, the following embodiments are given only for the purpose of explaining the present disclosure and are not intended to limit the present disclosure to the following contents.

A labeling method according to an embodiment has a step of labeling a substrate having a carbon-hydrogen bond with an oxygen isotope by using a catalyst and an oxidant produced from a hypervalent iodine compound having an ester structure and labeled water labeled with at least one oxygen isotope selected from the group consisting of ¹⁷O and ¹⁸O. According to this labeling method, a substrate having a carbon-hydrogen bond can be labeled by means of at least one selected from the group consisting of oxygen-17 isotope (¹⁷O) and oxygen-18 isotope (¹⁸O). The substrate may be labeled by means of either one of oxygen-17 isotope (¹⁷O) and oxygen-18 isotope (¹⁸O). That is, this labeling method is a labeling method using an oxygen isotope.

Labeling using an oxygen isotope according to the present disclosure is carried out by means of oxygen-17 isotope (¹⁷O) and/or oxygen-18 isotope (¹⁸O)). The labeling ratio (enrichment) according to the present disclosure is the proportion in which specific oxygen atoms constituting a compound are ¹⁷O and/or ¹⁸O. The labeling ratio of the labeled compound obtainable by the present labeling method may be 100% or less. In the present disclosure, when the proportion of ¹⁷O and/or ¹⁸O in the specific oxygen atoms constituting the compound is higher than the natural abundance ratio of oxygen isotopes (¹⁶O:¹⁷O:¹⁸O=99.759 atom %: 0.037 atom %: 0.204 atom %), it can be said that the compound is labeled. The labeling ratio according to the present disclosure is calculated by comparing the calculated values of the spectrum of a compound in which the isotope ratio of oxygen atoms measured by using a time-of-flight mass spectrometer is the natural abundance ratio, and the spectrum of the compound when all of the oxygen atoms are ¹⁸O and/or ¹⁷O.

As the catalyst, an oxidation catalyst can be used. Examples of such a catalyst include a metal complex and an enzyme. Examples of the metal complex include a metal complex of porphyrin and a metal complex of salen. The enzyme may be an oxidative enzyme, and specific examples include non-heme iron enzymes such as cytochrome P450 and lipoxygenase. Among these, it is preferable that the catalyst includes a metal complex, it is more preferable that the catalyst includes a ruthenium complex, and it is even more preferable that the catalyst includes at least one ruthenium complex selected from the group consisting of the following General Formulas (1), (2), and (3). A catalyst including such a ruthenium complex has excellent activity for an oxidation reaction of a carbon-hydrogen bond and also has excellent regioselectivity. Therefore, a compound labeled with an intended oxygen isotope can be obtained in high yield from various substrates having a carbon-hydrogen bond.

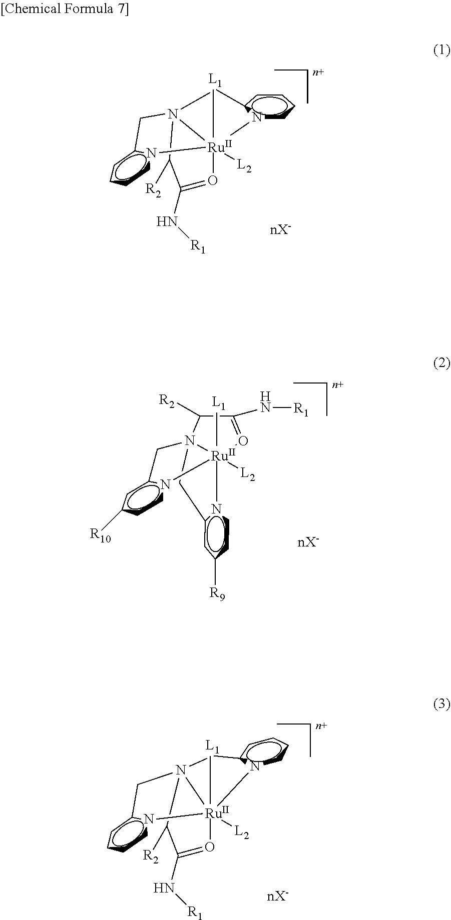

In the General Formulas (1), (2), and (3), R₁ represents a hydrogen atom, a phenyl group, or a monovalent group in which at least one hydrogen atom of a phenyl group is substituted with an alkyl group, a hydroxy group, a phenyl group, a halogen atom, or an alkoxy group. Among these, from the viewpoint of sufficiently increasing the activity and selectivity as a catalyst, R₁ is preferably a phenyl group or a monovalent group in which at least one hydrogen atom of a phenyl group (hydrogen atom on the benzene ring) is substituted with an alkyl group, a hydroxy group, a phenyl group, a halogen atom, or an alkoxy group. The monovalent group in which at least one hydrogen atom of a phenyl group is substituted with an alkyl group, a hydroxy group, a phenyl group, a halogen atom, or an alkoxy group can also be referred to as a substituted phenyl group.

When R₁ is a substituted phenyl group, the substituents substituting a plurality of hydrogen atoms in a phenyl group may be different from or identical with each other. The alkyl group substituting at least one hydrogen atom of the phenyl group may be a methyl group, an ethyl group, or a propyl group. The halogen atom substituting at least one hydrogen atom of the phenyl group may be a chlorine atom. The alkoxy group substituting at least one hydrogen atom of the phenyl group may be a methoxy group, an ethoxy group, or a propoxy group.

In the General Formulas (1), (2), and (3), R₂ represents a hydrogen atom, a phenyl group, or an alkyl group. When R₂ is an alkyl group, the alkyl group may be a methyl group, an ethyl group, or a propyl group. Among these, from the viewpoint of sufficiently increasing the activity and selectivity as the catalyst, it is preferable that R₂ is a hydrogen atom.

In the General Formula (3), R₉ and R₁₀ may be each independently a hydrogen atom, a halogen atom, or an alkyl group. The alkyl group may have 1 to 4 carbon atoms or may have 1 to 3 carbon atoms. Among these, from the viewpoint of sufficiently increasing the activity and selectivity as the catalyst, R₉ and R₁₀ may be each independently a halogen atom or an alkyl group, and R₉ and R₁₀ may be halogen atoms. The halogen atom may be a chlorine atom or a bromine atom. When R₉ and R₁₀ are halogen atoms, the halogen atom may be the same halogen atom as X, or may be a halogen atom different from X.

In the General Formulas (1), (2), and (3), L₁ represents a halogen atom or a water molecule. Among these, from the viewpoint of sufficiently increasing the activity and selectivity as the catalyst, it is preferable that L₁ is a halogen atom. In the General Formulas (1), (2), and (3), L₂ represents triphenylphosphine, pyridine, imidazole, or dimethyl sulfoxide. Among these, from the viewpoint of sufficiently increasing the activity and selectivity as the catalyst, it is preferable that L₂ is triphenylphosphine. In the General Formulas (1), (2), and (3), X represents a halogen atom. This halogen atom constitutes the ruthenium complex as an ion. X is, for example, a chlorine atom. In the General Formulas (1), (2), and (3), n represents 1 or 2. The oxidation number of Ru in the General Formulas (1), (2), and (3) is +2.

The above-described ruthenium complexes are useful as, for example, catalysts oxidizing a carbon-hydrogen bond. That is, these ruthenium complexes function as, for example, catalysts oxidizing a substrate having a carbon atom-hydrogen atom bond. For example, an oxygen-containing compound can be produced by oxidizing the substrate having the above-described bond. Examples of the oxygen-containing compound include a hydroxy compound and an oxo compound. In a hydroxy compound, an oxygen atom in a hydroxy group may be labeled with ¹⁷O or ¹⁸O. In an oxo compound, an oxygen atom of an oxo group may be labeled with ¹⁷O or ¹⁸O. The oxygen-containing compound may be a carbonyl compound having a carbonyl group or may be a ketone compound having a ketone group. Even in these cases, an oxygen atom in the carbonyl group and the ketone group may be labeled with ¹⁷O or ¹⁸O.

The oxidant is produced by using a hypervalent iodine compound having an ester structure and labeled water labeled with at least one oxygen isotope selected from the group consisting of ¹⁷O or ¹⁸O, as oxidant raw materials. Such an oxidant can be referred to as an oxidant for labeling using an oxygen isotope. When such an oxidant for labeling is used, an oxygen-containing compound obtainable by oxidizing a substrate having a carbon atom-hydrogen atom bond, is labeled with at least one oxygen isotope selected from the group consisting of ¹⁷O and ¹⁸O. A production method for the oxidant for labeling may have a step of reacting the hypervalent iodine compound with the labeled water.

Labeling using the oxygen isotope is carried out in the co-presence of the catalyst and the oxidant produced from a hypervalent iodine compound having an ester structure and labeled water. The hypervalent iodine compound having an ester structure can activate the labeled water in the reaction system. For this reason, the hypervalent iodine compound has a function of promoting oxidation of the substrate using ¹⁷O or ¹⁸O in the co-presence of a catalyst. From the viewpoint of further promoting such a function, the hypervalent iodine compound having an ester structure may have one aromatic ring or a plurality of aromatic rings. Since such a hypervalent iodine compound has strong electron donating properties, it can promote oxidation of a substrate having electron withdrawing properties. The hypervalent iodine compound having an ester structure, which is an oxidant raw material, may include a compound represented by the following General Formula (4)

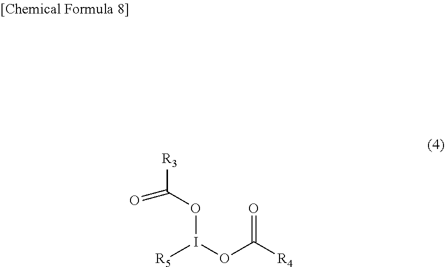

In the General Formula (4), R₃ and R₄ each independently represent a hydrogen atom, an alkyl group, or a monovalent group having an aromatic ring. R₅ represents a monovalent group having an aromatic ring. When R₃ and R₄ are alkyl groups, at least one hydrogen atom of the alkyl group may be substituted with a functional group. R₃, R₄, and R₅ may each have a benzene ring. R₃, R₄, and R₅ may be each independently an unsubstituted phenyl group or a substituted phenyl group having at least one hydrogen atom substituted. Examples of the substituted phenyl group include those in which at least one hydrogen atom in the phenyl group (benzene ring) is substituted with a heteroatom, a halogen atom, a hydroxy group, a nitro group, or an organic group different from these. At least one hydrogen on the aromatic ring in at least one of R₃, R₄, and R₅ in the General Formula (4) may be substituted with a halogen atom. From the viewpoint of sufficiently increasing the labeling ratio using an oxygen isotope, for example, the hypervalent iodine compound may include a compound represented by the following General Formula (5).

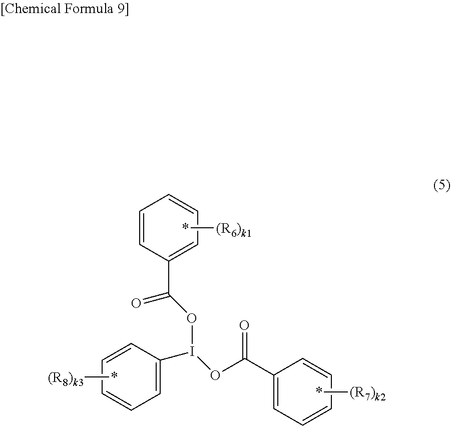

In the General Formula (5), R₆, R₇, and R₈ each represent a heteroatom, and k1, k2, and k3 each represent an integer of 0 to 5. A coupling end represented by the symbol * in R₆, R₇, and R₈ is bonded to a carbon atom constituting the benzene ring and substitutes a hydrogen atom in the benzene ring. When the compound has a plurality of R₆'s, multiple R₆'s may be identical with each other or may be different from each other. When the compound has a plurality of R₇'s, multiple R₇'s may be identical with each other or may be different from each other. When the compound has a plurality of R₈'s, multiple R₈'s may be identical with each other or may be different from each other. R₆, R₇, and R₈ may be identical with each other or may be different from each other. k1, k2, and k3 may be identical with each other or may be different from each other. From the viewpoint of sufficiently increasing the labeling ratio using an oxygen isotope, R₆ and R₇ may be each a halogeno group. The halogeno group for R₆ and R₇ may be each independently a fluoro group (—F) or may be a chloro group (—Cl). At this time, k1 and k2 may be each independently 1 to 5, may be 2 to 5, or may be 3 to 5. Furthermore, k3 may be 0.

In the above-described step, for example, the oxidant produced in the system from the hypervalent iodine compound having an ester structure and the labeled water in the presence of the catalyst including the ruthenium complex, oxidizes a carbon-hydrogen bond in the substrate to obtain the oxygen-containing compound in which a hydroxy group or an oxo group is bonded to a carbon atom. The oxygen-containing compound may include at least one of an alcohol, a ketone, and an aldehyde. Regarding the mechanism of the reaction, it is speculated that when carbon atoms of the substrate are oxidized with the produced oxidant, the substrate is labeled with ¹⁷O or ¹⁸O. A labeled compound labeled with ¹⁷O or ¹⁸O (labeled oxygen-containing compound) can be obtained by the above-described step. Regarding the labeled water, commercially available oxygen-17-labeled water or oxygen-18-labeled water can be used. Furthermore, if necessary, the substrate may be labeled with both ¹⁷O and ¹⁸O by using mixed labeled water of oxygen-17-labeled water and oxygen-18-labeled water. The amount of use of the labeled water may be 1 to 10 equivalents, or may be 1 to 4 equivalents, with respect to the substrate. Even when the amount of use of the labeled water is reduced in this way, the labeling ratio can be sufficiently increased. The labeling ratio using at least one selected from the group consisting of ¹⁷O and ¹⁸O may be 60 atom % or more, may be 80 atom % or more, or may be 90 atom % or more.

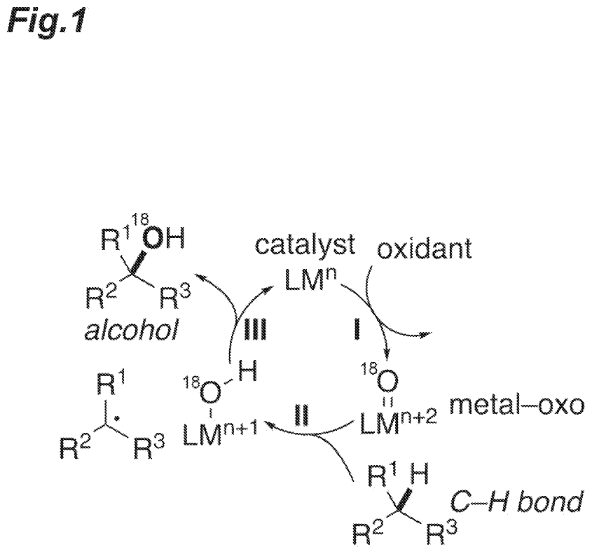

FIG. 1 shows an example of the mechanism of producing an alcohol, which is one kind of hydroxy compounds, from a substrate including tertiary carbon atoms by using the above-mentioned ruthenium complex as an oxidation catalyst. Incidentally, the mechanism for alcohol production is not limited to this example.

In step I in FIG. 1, an oxidant produced from a hypervalent iodine compound having an ester structure and labeled water is brought into contact with a ruthenium complex (LMₙ: L represents a ligand, M represents ruthenium, and n represents 1 or 2) to form a ruthenium-oxo bond. Tetrachloroethane can be used as the solvent. From the viewpoint of improving the catalytic activity and the selectivity of the position to be oxidized (regioselectivity) in the substrate, an acid may be used together with the oxidant. Examples of the acid include acetic acid, monochloroacetic acid, dichloroacetic acid, trichloroacetic acid, trifluoroacetic acid, and pentafluorobenzoic acid.

In step II, the ruthenium-oxo bond and the substrate including a tertiary carbon atom are brought into contact with each other, a hydrogen atom is eliminated from the substrate, and a substrate radical and a ruthenium-hydroxy bond (including ¹⁸O) are produced. R¹, R², and R³ in the substrate may be different from each other or may be identical with each other. At least two selected from the group consisting of R¹, R², and R³ may be bonded to form a ring.

The substrate may be a hydrocarbon having 5 to 30 carbon atoms, in which at least one hydrogen atom is substituted with a functional group, an oxygen-containing hydrocarbon, or a sugar. The hydrocarbon may be any of a chain-like (linear or branched) hydrocarbon, an alicyclic hydrocarbon, and an aromatic hydrocarbon. Examples of the functional group include a hydroxy group, a halogen atom, an alkoxy group, an aldehyde group, an acyl group, a carboxyl group, an allyl group, an amino group, a nitro group, an acetyl group, an oxo group, and an ester group. The substrate may be a pro-hormone.

In step III, the hydroxy group (including ¹⁸O) bonded to ruthenium is bonded to the substrate radical to obtain a labeled compound. Thereafter, the ruthenium complex is utilized again as the catalyst. Furthermore, in FIG. 1, ¹⁸O is shown as the oxygen isotope; however, the compound may be labeled with ¹⁷O by using oxygen-17-labeled water. The compound may also be labeled with both ¹⁷O and ¹⁸O by using mixed labeled water of oxygen-17-labeled water and oxygen-18-labeled water. Incidentally, a hydroxy compound is obtained in this example; however, another compound (for example, an oxo compound) may be obtained by changing the substrate or adjusting the reaction conditions.

The reaction mechanism in the step of labeling with an oxygen isotope according to the present embodiment is not limited to that of FIG. 1. For example, when the substrate includes a sugar, this sugar may be any of a monosaccharide, a disaccharide, and a polysaccharide. Examples of the monosaccharide include a triose, a tetrose, a pentose, and a hexose (hexose). The hexose may be an aldohexose or may be a ketohexose. Examples include allose, altrose, glucose, mannose, gulose, idose, galactose, talose, psicose, fructose, sorbose, and tagatose. Each of the sugars may either a stereoisomer or an optical isomer. A substrate including any one of these can be labeled by the labeling method of the present embodiment.

For example, D-mannose can be labeled with ¹⁸O by the following scheme as shown in the Examples. In the following scheme, an example of labeling a hydroxy group at the 3-position with ¹⁸O is described; however, D-mannose may be labeled with ¹⁷O or both ¹⁷O and ¹⁸O in the same manner as that example.

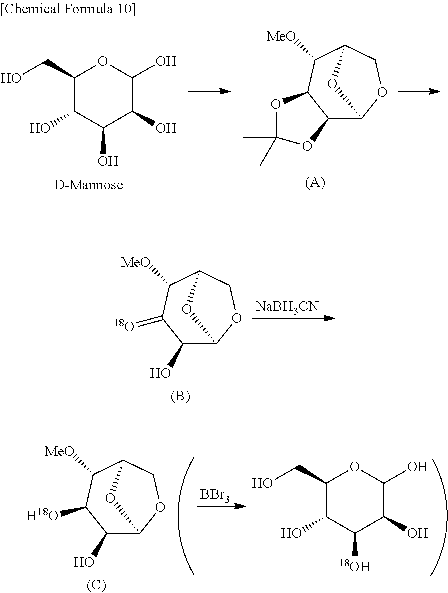

Examples of a mannose derivative derived from D-mannose include compounds of (A), (B), and (C) in the above-described scheme. The compound of (A) (1,6-anhydro-4-O)-methyl-2,3-O-isopropylidene-β-D-mannopyranose), the compound (B) (1R,2R,4R,5R)-4-hydroxy-2-methoxy-6,8-dioxabicyclo[3.2.1]-octan-3-one), and the compound of (C) (1,6-anhydro-4-O)-methyl-β-D-mannopyranose) are all compounds useful for obtaining D-mannose labeled with an oxygen isotope (O¹⁷O or ¹⁸O). Incidentally, the compound of (C) has superior stability compared to the compound of (B). For this reason, the labeling ratio of D-mannose using an oxygen isotope can be increased by conducting synthesis of the mannose derivative (C) from the mannose derivative (A) by a one-pot reaction.

Among the above-mentioned mannose derivatives (A), (B), and (C), the compound of (B) does not have to be labeled with an oxygen isotope. A compound that is not labeled with an oxygen isotope [(1R,2R,4R,5R)-4-hydroxy-2-methoxy-6,8-dioxabicyclo[3.2.1]octan-3-one)] is represented by the following Formula (8) (wherein Me represents a methyl group). This compound can be used as, for example, a reagent. D-mannose may be synthesized by using this compound. The compound of Formula (8) can be synthesized from the derivative (A) not by using labeled water but by using ordinary water.

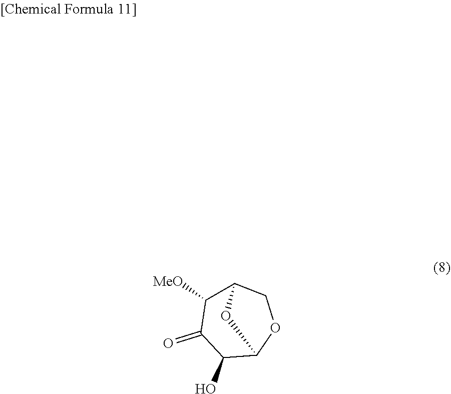 [SMILES: CO[C@H]1C(=O)[C@H](O)[C@@H]2OC[C@H]1O2]

According to the labeling method (labeling method) of the present embodiment, an intended oxygen isotope-labeled compound can be obtained in high yield while reducing the amount of use of labeled water. For this reason, labeled water, which is rare, can be effectively utilized. The labeled compound thus obtained can be used as a labeled molecular probe. For example, the labeled compound can be used for measurement by ¹⁷O nuclear magnetic resonance spectroscopy (NMR) and imaging (MRI). Objects such as cells can be visualized by such measurement. Furthermore, when a sugar labeled with the oxygen isotope is used, cellular tissue can be observed by means of an isotope microscope or the like. Thus, the labeling method (oxygen isotope labeling method) and the labeled compound (oxygen isotope-labeled compound) of the present embodiment can significantly expand the range of application of technologies for utilizing labels.

Next, an example of a production method for the above-mentioned ruthenium complex will be described below. The production method of this example has a first step of causing an amino acid ester compound represented by General Formula (iii), a methylpyridine compound represented by General Formula (iv), and an amine compound represented by General Formula (v) to react to synthesize a ligand.

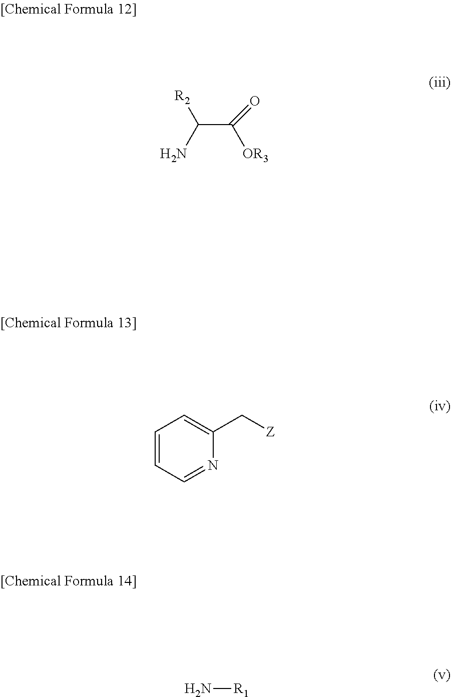

In General Formula (iii), R₂ is the same as R₂ in General Formulas (1), (2), and (3) of the above-mentioned ruthenium complex. In General Formula (iii), R₃ represents an alkyl group having 1 to 3 carbon atoms. R₃ is, for example, a methyl group. In General Formula (iv), Z represents a halogen atom. The methylpyridine compound is, for example, chloromethylpyridine. In General Formula (v), R₁ is the same as R₁ in General Formulas (1), (2), and (3) of the above-mentioned ruthenium complex.

In the first step, a ligand is obtained by performing N-alkylation of an amino acid ester (General Formula (iii)) using a methylpyridine compound (General Formula (iv)), hydrolysis of an amino acid ester, and amidation of an amine (General Formula (v)). This ligand is represented by the following General Formula (vi).

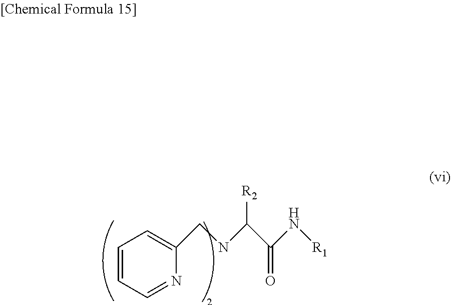

After the first step, a second step of reacting the above-described ligand with a ruthenium compound to obtain a ruthenium complex including at least one selected from the group consisting of a trans-form ruthenium complex represented by General Formula (1) and a cis-form ruthenium complex represented by General Formula (2), is carried out.

A complex having ruthenium(II) chloride as the ruthenium compound and dimethyl sulfoxide or triphenylphosphine as the ligand may be mentioned. Examples of such a complex include dichlorotetrakis(dimethyl sulfoxide)ruthenium(II) and tris(triphenylphosphine)ruthenium(II) dichloride. The second step may be carried out while heating under reflux by using an alcohol such as ethanol as a solvent. Ruthenium complexes represented by General Formulas (1) and (2) are obtained by such a step.

After the second step, a step of separating the trans-form ruthenium complex represented by General Formula (1) and the cis-form ruthenium complex represented by General Formula (2) may be carried out. This step may be carried out by, for example, column chromatography. As a result, the trans-form ruthenium complex represented by General Formula (1) and the cis-form ruthenium complex represented by General Formula (2) can be obtained. The ruthenium complex represented by General Formula (3) may be synthesized by a method similar to that for the ruthenium complexes of General Formulas (1) and (2) or may be synthesized by the method described in the Examples. The method described in the Examples may be appropriately changed based on the description given above.

Embodiments of the present disclosure have been described above; however, the present disclosure is not intended to be limited to the above-described embodiments.

### EXAMPLES

The contents of the present disclosure will be described in more detail with reference to the Examples; however, the present disclosure is not intended to be limited to the following Examples.

### Example 1-1

### <Synthesis of Ligand 1>

N-alkylation of an amino acid ester was carried out by performing a reaction of Reaction Formula (1a) by the following procedure. Incidentally, Me represents a methyl group.

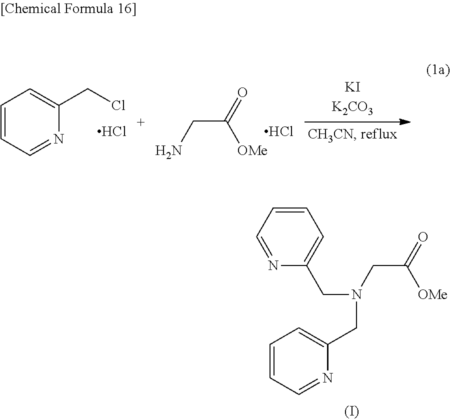

2.76 g (20 mmol) of potassium carbonate, 332 mg (2 mmol) of potassium iodide, and 10 mL of acetonitrile were introduced into a pear-shaped flask. To this, 251 mg (2 mmol) of glycine methyl ester hydrochloride and 820 mg (5 mmol) of 2-(chloromethyl)pyridine hydrochloride were added. The mixture was maintained for 24 hours under reflux conditions at 95° C. to obtain a reaction liquid. This was cooled to room temperature, subsequently light components were distilled off with a rotary evaporator, and then a product was obtained. 50 g of ethyl acetate was added to this product, and then filtration was performed to remove a solid material (salt). The filtrate was washed with an aqueous solution of sodium carbonate and sodium chloride, sodium sulfate was added to the washing liquid to remove moisture, and then light components were distilled off with a rotary evaporator. The obtained liquid product was separated and purified by column chromatography using basic silica gel (dichloromethane/methanol=100:1), and then light components were distilled off again with a rotary evaporator to obtain a product. This product was analyzed by ¹H-NMR, and as a result, a product of Formula (I) shown in Reaction Scheme (1a) (bis(pyridin-2-ylmethyl)glycine methyl ester, 0.4634 g, 1.71 mmol, yield 85%) was obtained.

Next, as shown in Reaction Scheme (1b), hydrolysis of the above-described product was performed. The details of the procedure are as follows.

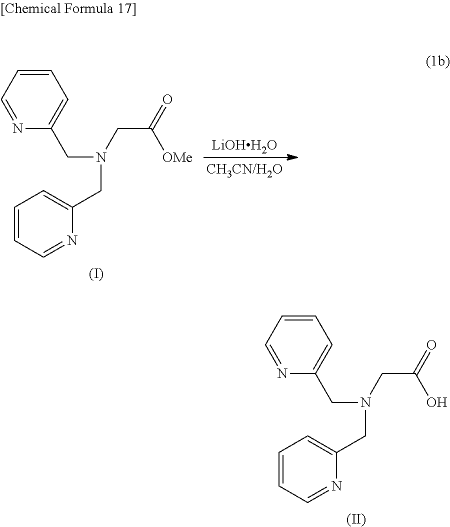

8.3 mL of a mixed solvent obtained by mixing acetonitrile and water at a mass ratio of 1:1, and 282.14 mg (1.04 mmol) of the product of Reaction Scheme (1a) were introduced into an eggplant-shaped flask, and a solution was obtained. To this solution, 52.36 mg (1.25 mmol) of lithium hydroxide monohydrate was added, and the mixture was stirred at room temperature for 4 hours. Subsequently, 20 mL of dichloromethane was incorporated, and an aqueous phase was collected. Hydrochloric acid was added to this aqueous phase to neutralize the aqueous phase, and the pH was adjusted to 6. Thereafter, a mixed solvent obtained by mixing trichloromethane and methanol at a mass ratio of 9:1 was incorporated, and an extract was obtained. Magnesium sulfate was added to this extract, and the mixture was dehydrated. Thereafter, light components were distilled off by using a rotary evaporator, and a liquid product was obtained. The liquid product was analyzed by ¹H-NMR and ¹³C-NMR, and it was confirmed that the liquid product was a compound represented by Formula (II) in the above-described Reaction Scheme (1b) (bis(pyridin-2-ylmethyl)glycine). The amount of production of this compound was 401.55 mg (1.561 mmol), and the yield was 94%.

Synthesis of a ligand by the following Reaction Scheme (1c) was performed in an atmosphere at room temperature. 678.7 mg (2.638 mmol) of bis(pyridin-2-ylmethyl)glycine obtained by the above-described Reaction Scheme (1b), 10 ml of 2-propanol, 270.2 mg (2.902 mmol) of aniline, and 762.2 mg (2.902 mmol) of 4-(4,6-dimethoxy-1,3,5-triazin-2-yl)-4-methylmorpholinium chloride were introduced into a round-bottom flask. Then, the mixture was stirred at room temperature for 20 hours. Filtration of the obtained mixed liquid was performed, and then light components were distilled off with a rotary evaporator to obtain a product.

Purification of the product was performed by using column chromatography (basic silica gel, hexane/ethyl acetate=4:1 to 0:1), and a purification product was obtained. Structure analysis was performed by ¹H-NMR and ¹³C-NMR, and as a result, it was confirmed that 2-(bis(pyridin-2-ylmethyl)amino)-N-phenylacetamide represented by Formula (E) of Reaction Scheme (1c) was obtained (yield quantity: 652.6 mg, 1.963 mmol, yield: 74.42%).

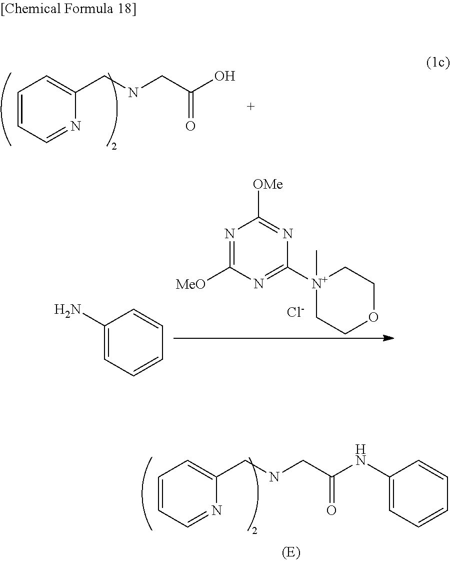

### <Synthesis of Metal Complex>

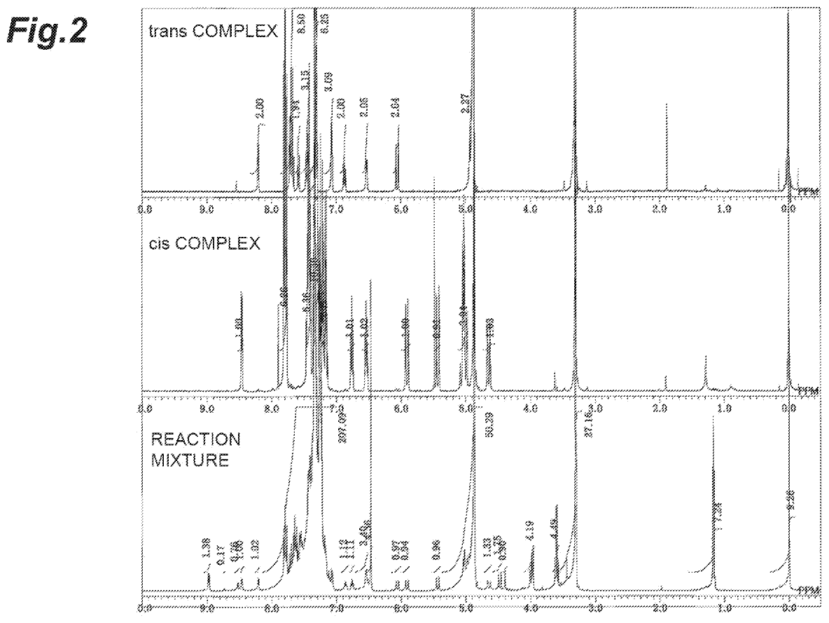

Synthesis of a ruthenium complex was performed by the following Reaction Scheme (2) under heating conditions at 95° C. in an air atmosphere. 33.2 mg (0.100 mmol) of 2-(bis(pyridin-2-ylmethyl)amino)-N-phenylacetamide synthesized by the above-described Reaction Scheme (1), 4 mL of ethanol, and 105 mg (110 μmol) of RuCl₂(PPh₃)₃ were introduced into a Schlenk tube (capacity: 10 ml) dried in an oven. Reaction was carried out for 4 hours under reflux conditions at 95° C., and a reaction mixture was obtained. The reaction mixture was filtered, and then the filtrate was concentrated under reduced pressure to obtain a concentrate. Thereafter, separation of the concentrate was performed by two-step column chromatography. The ¹H-NMR measurement results for the reaction mixture after filtration were as shown at the bottom of FIG. 2.

As a first stage, a mixture of a trans-form ruthenium complex and a cis-form ruthenium complex was obtained by using a neutral silica gel column (dichloromethane/methanol=19/1 to 4/1). As a second stage, the trans-form ruthenium complex and the cis-form ruthenium complex were separated by using chromatography using neutral silica gel (chloroform/methanol=14/1 to 4/1).

¹H-NMR and ¹³C-NMR measurement and single crystal X-ray structural analysis of the two obtained products were carried out, and as a result, a trans-form ruthenium complex represented by Formula [III] and a cis-form ruthenium complex represented by Formula [IV] of the following Reaction Scheme (2) were obtained (wherein R═C₆H₅).

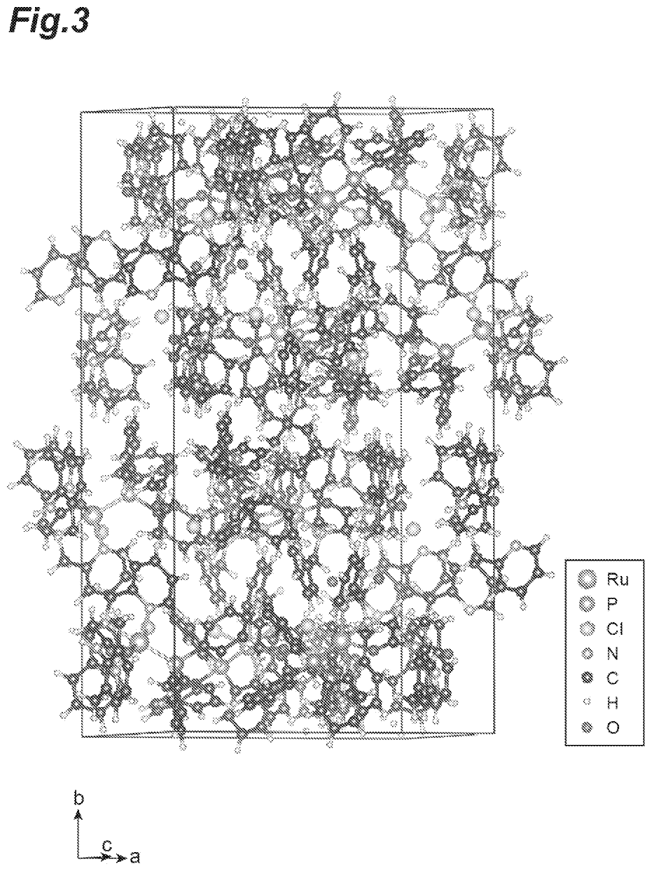

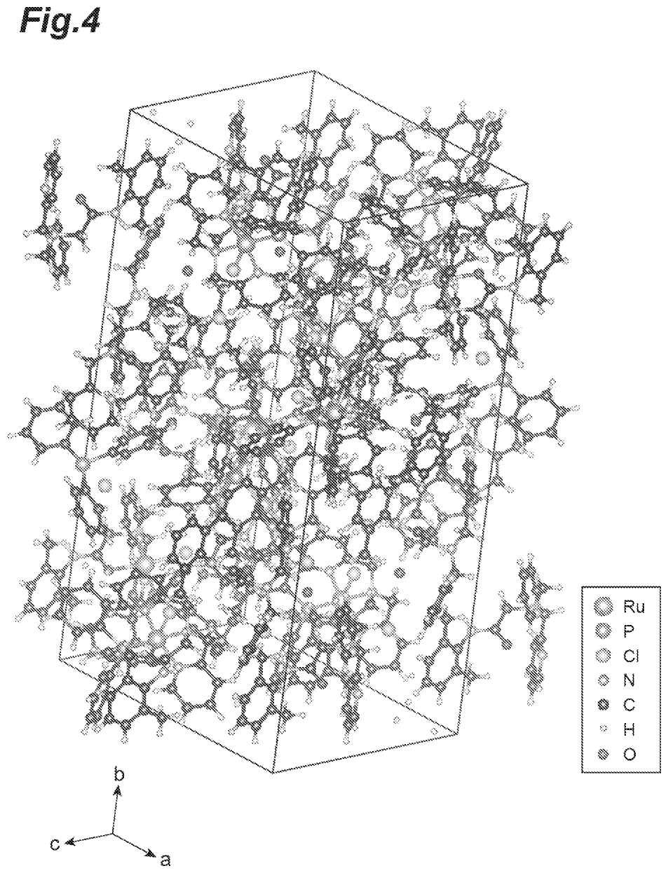

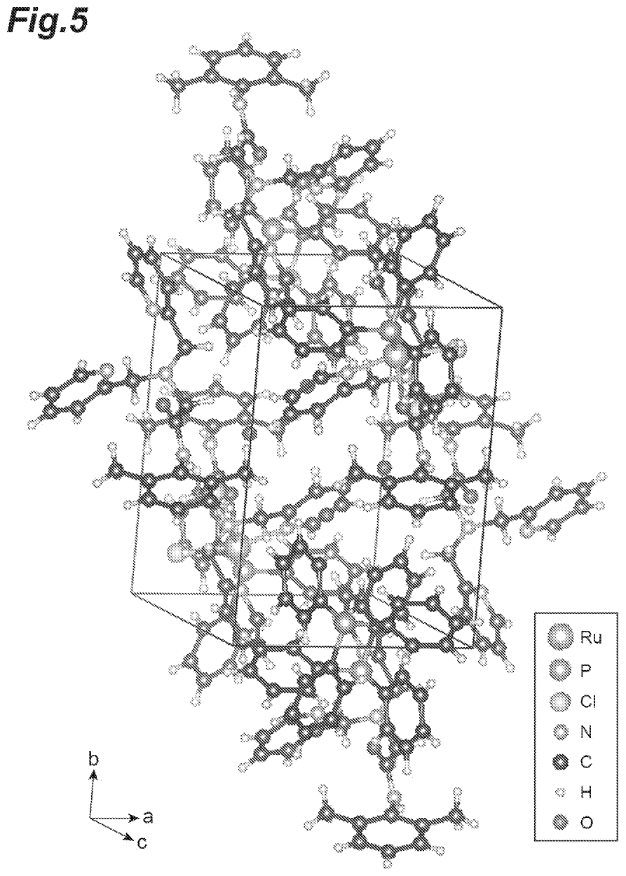

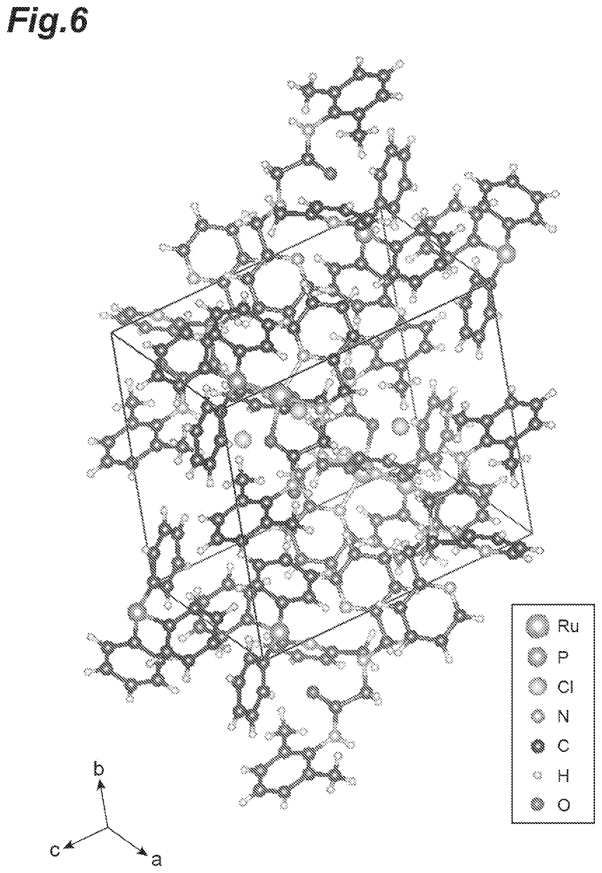

The ¹H-NMR measurement results for the trans-form ruthenium complex were as shown at the top of FIG. 2. The ¹H-NMR measurement results for the cis-form ruthenium complex were as shown in the middle of FIG. 2. FIG. 3 is a diagram showing the results of a single crystal structural analysis of the trans-form ruthenium complex. FIG. 4 is a diagram showing the results of a single crystal structure analysis of the trans-form ruthenium complex viewed from an angle different from that of FIG. 3. FIG. 5 is a diagram showing the results of a single crystal structure analysis of a cis-form ruthenium complex. FIG. 6 is a diagram showing the results of a single crystal structure analysis of the cis-form ruthenium complex viewed from an angle different from that of FIG. 5.

The yield quantity of the trans-form ruthenium complex (MW: 766.09690) was 17.6 mg (23.0 μmol), and the yield was 23.0%. On the other hand, the yield quantity of the cis-form ruthenium complex (MW: 766.09690) was 28.8 mg (37.6 μmol), and the yield was 37.6%.

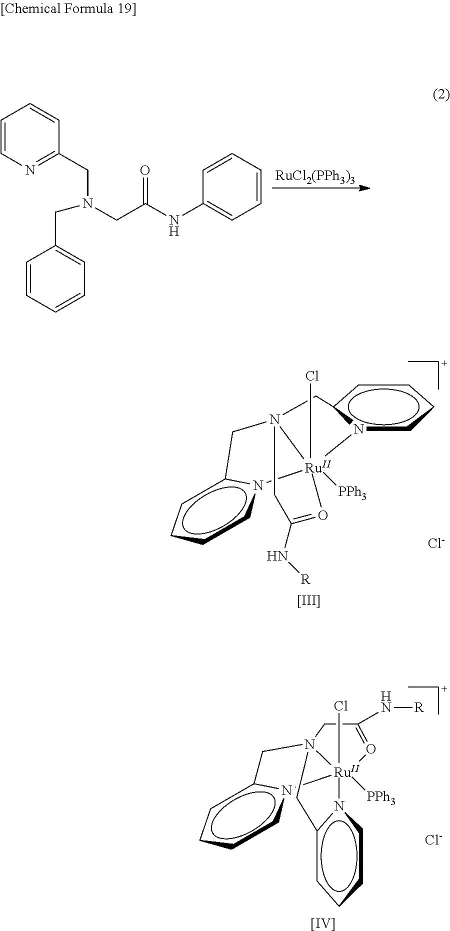

### Example 1-2

### Synthesis of N-2,6-dimethylphenyl-2-chloroacetamide

Chloroacetic acid (1.42 g, 15 mmol), 2,6-dimethylaniline (2.0 mL, 16.5 mmol), and 4-dimethylaminopyridine (183 mg, 1.5 mmol) were introduced into dichloromethane (60 mL) in a container, and the mixture was cooled to 0° C. by using an ice bath. After cooling, dicyclohexylcarbodiimide (3.43 g, 16.5 mmol) was introduced into the container, subsequently the ice bath was removed, and stirring was performed for 3 hours while returning the temperature to room temperature. The obtained reaction liquid was eluted with basic silica gel (ethyl acetate) to obtain a solution. The solvent was distilled off from the solution by using a rotary evaporator, and a crude product was obtained.

The crude product was purified by column chromatography (hexane/ether=4/1) using basic silica gel, and the solvent was distilled off by using a rotary evaporator. The obtained product was analyzed by ¹H-NMR, and it was confirmed that N-2,6-dimethylphenyl-2-chloroacetamide (1.75 g, 8.85 mmol, yield: 59%) was produced.

### Synthesis of 2-(2-pyridyl)-N-(2-pyridylmethyl)ethyl-1-amine

1.22 g (10 mmol) of 2-(2-pyridyl)ethylamine and 1.07 g (10 mmol) of 2-pyridyl carboxaldehyde were dissolved in 10 mL of ethanol, and 45.4 mg (12 mmol) of sodium borohydride was added thereto. Subsequently, stirring was performed for 3 hours while heating under reflux. Subsequently, the mixture was cooled to room temperature, and 15 mL of a saturated aqueous solution of sodium hydrogen carbonate was added thereto to obtain a solution. An operation of extracting the solution with 5 mL of ethyl acetate was repeatedly carried out three times, and then concentration under reduced pressure was carried out with a rotary evaporator to obtain a reaction mixture. The reaction mixture was purified by column chromatography (mixed solution of hexane/ethyl acetate=1/1) using basic silica gel, and then the solvent was distilled off by using a rotary evaporator. The obtained product was analyzed by ¹H-NMR, and it was confirmed that the product was 2-(2-pyridyl)-N-(2-pyridylmethyl)-ethyl-1-amine (1.58 g, 7.42 mmol, yield: 74%) (see J. Am. Chem. Soc., 2018, 140, 1, 58-61).

### <Synthesis of Metal Complex>

N-2,6-dimethylphenyl-2-chloroacetamide (593 mg, 3 mmol) prepared as described above, 2-(2-pyridyl)-N-(2-pyridylmethyl)ethyl-1-amine (640 mg, 3 mmol), potassium carbonate (622 mg, 4.5 mmol), and potassium iodide (598 mg, 3 mmol) were added to 20 mL of acetonitrile, and stirring was performed for 3 hours while heating under reflux to obtain a reaction solution. The solvent was distilled off from the reaction solution by using a rotary evaporator, dichloromethane was added thereto, and the mixture was washed with a saturated aqueous solution of sodium hydrogen carbonate (20 mL) and a saturated aqueous solution of sodium chloride (20 mL). The obtained solution was dried by using sodium sulfate, the solvent was distilled off by using a rotary evaporator, and a reaction mixture was obtained. The reaction mixture was purified by column chromatography (basic silica gel, hexane/ethyl acetate=2/1), and the solvent was distilled off by using a rotary evaporator to obtain a product. It was confirmed by ¹H-NMR analysis that this product was N′-2,6-dimethylphenyl-N-2-(2-pyridyl)ethyl, N-2-(2-pyridyl)methylacetamide (1.03 g, 2.8 mmol, yield: 92%).

N′-2,6-dimethylphenyl-N-2-(2-pyridyl)ethyl, N-2-(2-pyridyl)methylacetamide (820 mg, 2.2 mmol) thus obtained and RuCl₂(PPh₃)₃ (2.3 g, 2.41 mmol) were added to 90 mL of ethanol, and stirring was performed for 12 hours while heating under reflux to obtain a reaction liquid. Celite filtration of this reaction liquid was performed to remove solid components, and then concentration under reduced pressure was carried out by using a rotary evaporator to obtain a reaction mixture. The reaction mixture was purified by column chromatography (neutral silica gel, chloroform/methanol=12/1 to 4/1), and the solvent was distilled off with a rotary evaporator. It could be confirmed by a single crystal X-ray structure analysis that the obtained product was a ruthenium complex of the following Formula (V) (290 mg, 0.36 mmol, yield: 16.4%).

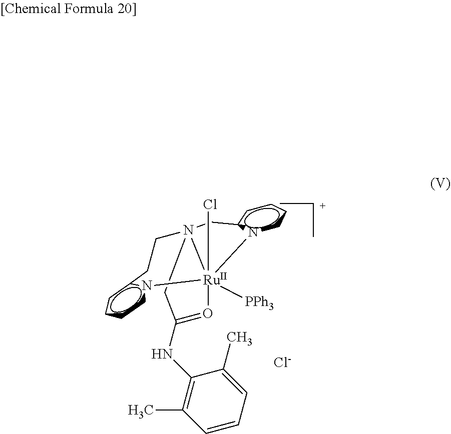

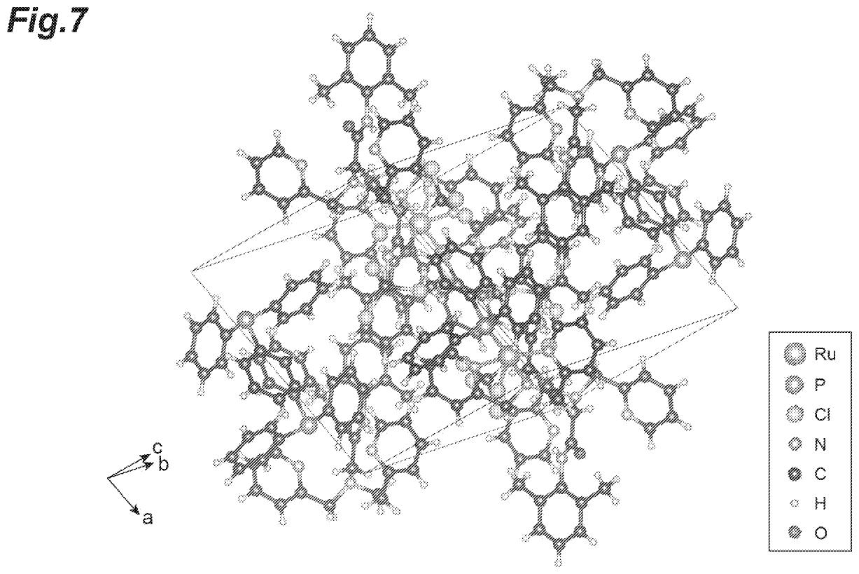

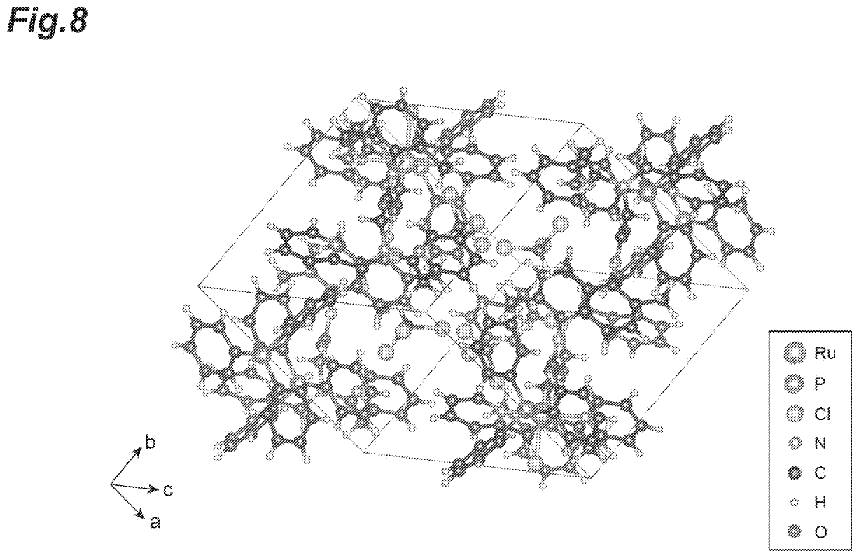

FIG. 7 is a diagram showing the results of a single crystal structure analysis of the ruthenium complex of Formula (V). FIG. 8 is a diagram showing the results of a single crystal structure analysis of the ruthenium complex of Formula (V) as viewed from an angle different from that of FIG. 7.

### Example 1-3

### <Synthesis of Oxidant Raw Material>

A reaction of the following Formula (1-3) was carried out by referring to a known synthesis method (Angew. Chem. Int. Ed. 2014, 53, 11060-11064) in air at 45° C. Specifically, 3.22 g (10.0 mmol, 1.0 equivalent) of (diacetoxyiodo)benzene, 4.24 g (20.0 mmol, 2.0 equivalents) of pentafluorobenzoic acid, 100 mL of dichloromethane, and 100 mL of toluene were introduced into a 300-mL eggplant-shaped flask. The obtained reaction solution was concentrated under reduced pressure at 45° C. by using a rotary evaporator, and the solvent was distilled off. An operation of adding 50 mL of toluene to the obtained reaction mixture and concentrating the reaction mixture under reduced pressure was carried out four times by using a rotary evaporator to obtain a mixture. Recrystallization of the mixture was performed by using hexane/dichloromethane, and the generated crystals were collected by filtration, washed with hexane, and dried under reduced pressure. The obtained product was subjected to ¹H-NMR and ¹³C-NMR to analyze the obtained product, and it was confirmed that 5.20 g (8.30 mmol) of [bis(pentafluorobenzoyloxy)iodo]benzene (yield: 83.0%, (VI) of the following Formula (1-3)) was obtained.

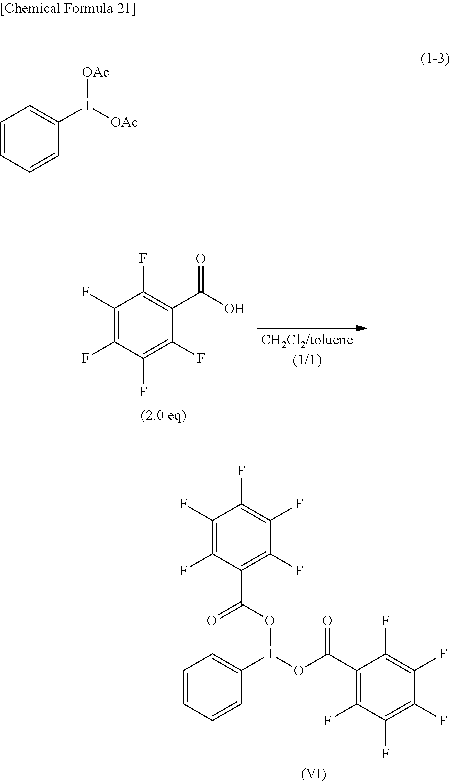 [SMILES: CC(=O)OI(OC(C)=O)c1ccccc1 ; O=C(O)c1c(F)c(F)c(F)c(F)c1F ; O=C(OI(OC(=O)c1c(F)c(F)c(F)c(F)c1F)c1ccccc1)c1c(F)c(F)c(F)c(F)c1F ; "(VI)"]

### Example 2-1

### <Hydroxylation>

Hydroxylation of adamantane represented by the following Formula (2-1) was carried out by using the ruthenium complex of Formula [IV] obtained according to Example 1-1 (wherein R in Formula [IV] is a 2,6-dimethylphenyl group) as a catalyst, under a nitrogen gas stream at a reaction temperature of 35° C. Specifically, 27.2 mg (0.20 mmol) of adamantane and 3.18 mg (4.0 μmol) of the ruthenium complex of Formula [IV] were introduced into a 5-mL Schlenk tube. The interior of this Schlenk tube was purged with nitrogen, and then 0.5 mL of 1,1,2,2-tetrachloroethane and 8.0 mg (7.2 μL, 0.40 mmol, 2 equivalents with respect to the substrate) of ¹⁸O-labeled water (oxygen-18 isotope-labeled water, ≥98 atom % 180) were introduced into the Schlenk tube. In addition, the oxidant raw material of Formula (VI) (250 mg, 0.40 mmol) obtained in Example 1-3 was added thereto, and stirring was performed at 35° C. for 0.5 hours.

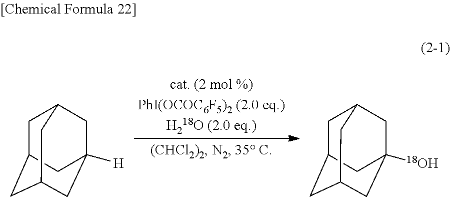 [SMILES: C1C2CC3CC1CC(C2)C3 ; [18OH]C12CC3CC(CC(C3)C1)C2]

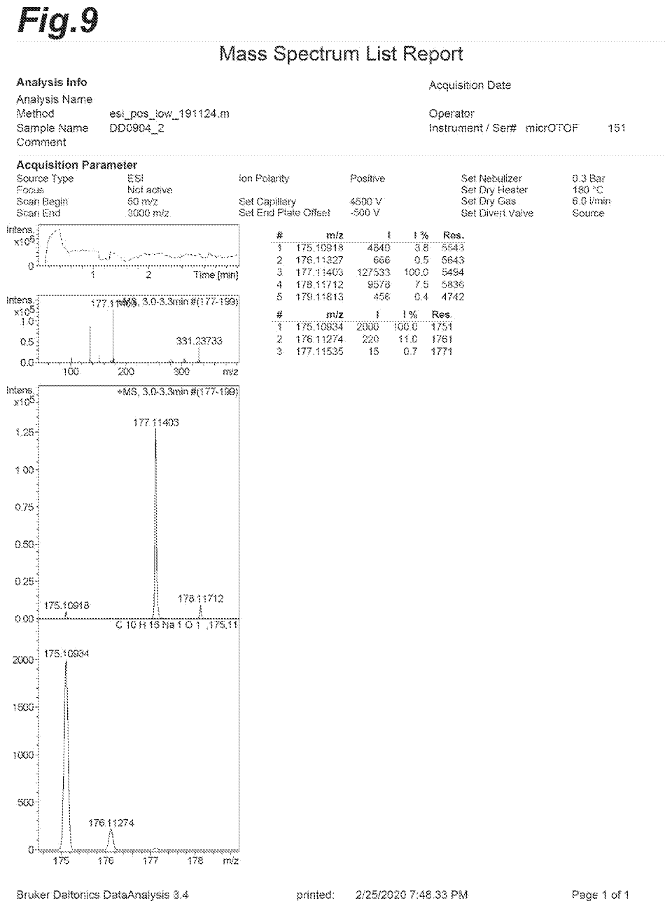

After completion of stirring, carboxylic acids and the catalyst were removed from the reaction mixture by using short column chromatography (basic silica gel, developing solvent: ethyl acetate), and then the solvent was distilled off by using a rotary evaporator. The obtained mixture was purified by using column chromatography (neutral silica gel, developing solvent: hexane/ethyl acetate=9/1 to 2/1), and then the solvent was distilled off by using a rotary evaporator. The obtained product was analyzed by ¹H-NMR, and it was confirmed that the target product adamantan-1-ol (21.9 mg, yield: 71%) was obtained. Analysis was performed with a time-of-flight mass spectrometer (ESI-TOF-MS), and it was confirmed that the labeling ratio (enrichment factor) with oxygen-18 isotope (¹⁸O)) was 96 atom %. FIG. 9 shows the analysis results obtained using a time-of-flight mass spectrometer.

### Example 2-2

### <Hydroxylation>

Hydroxylation of 3,7-dimethyloctyl acetate was carried out according to the following Formula (2-2) by using the ruthenium complex of Formula [IV] (wherein R in Formula [IV] is a 2,6-dimethylphenyl group) obtained according to Example 1-1 as a catalyst, under a nitrogen gas stream at a reaction temperature of 35° C. Specifically, the ruthenium complex of Formula [IV] (3.18 mg, 4.0 μmol) was introduced into a 5-mL Schlenk tube, the interior of the Schlenk tube was purged with nitrogen, and then 0.5 mL of 1,1,2,2-tetrachloroethane, 40.1 mg (0.20 mmol) of 3,7-dimethyloctyl acetate, and 8.0 mg (7.2 μL, 0.40 mmol, 2 equivalents with respect to the substrate) of ¹⁸O-labeled water (≥98 atom %) were introduced into the Schlenk tube. In addition, 250 mg (0.40 mmol) of the oxidant raw material of Formula (VI) obtained in Example 1-3 was added thereto, and stirring was performed at 35° C. for 12 hours.

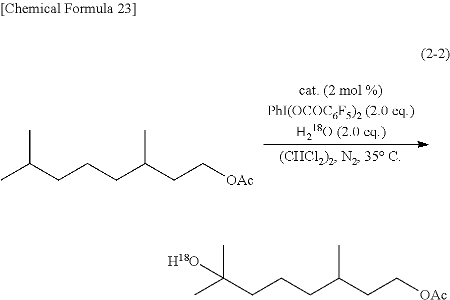

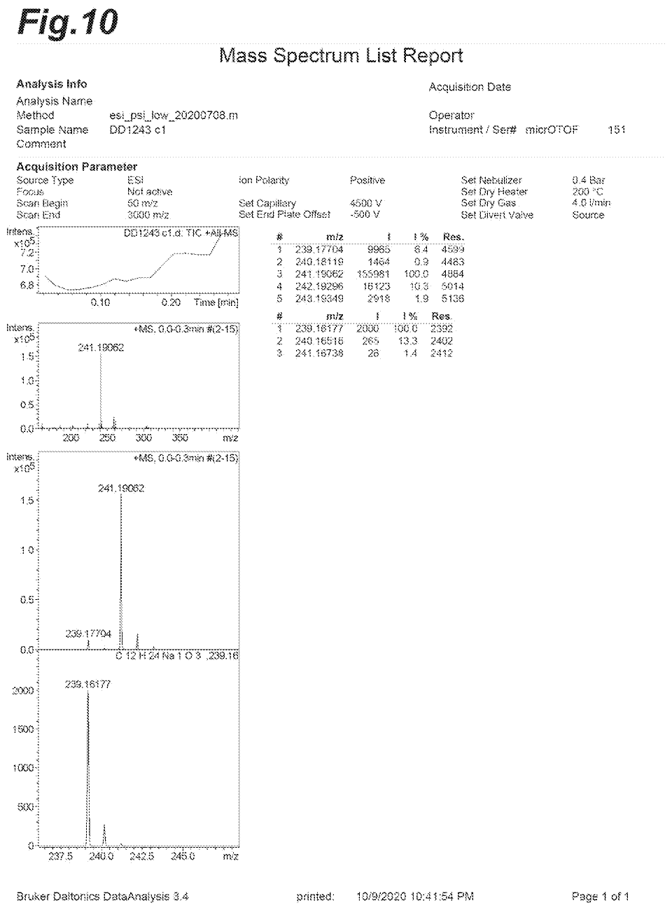

After completion of stirring, carboxylic acids and the catalyst were removed from the reaction mixture by using short column chromatography (basic silica gel, developing solvent: ethyl acetate), and then the solvent was distilled off by using a rotary evaporator. The obtained mixture was purified by using column chromatography (neutral silica gel, developing solvent: hexane/ethyl acetate=9/1 to 2/1), and then the solvent was distilled off by using a rotary evaporator. The obtained product was analyzed by ¹H-NMR, and it was confirmed that the target product 7-hydroxy-3,7-dimethyloctyl acetate (30.1 mg, yield: 69%) was obtained. Analysis was performed with a time-of-flight mass spectrometer (ESI-TOF-MS), and it was confirmed that the labeling ratio (enrichment factor) with oxygen-18 isotope (¹⁸O) was 94 atom %. FIG. 10 shows the analysis results obtained using a time-of-flight mass spectrometer.

### Example 2-3

### <Hydroxylation>

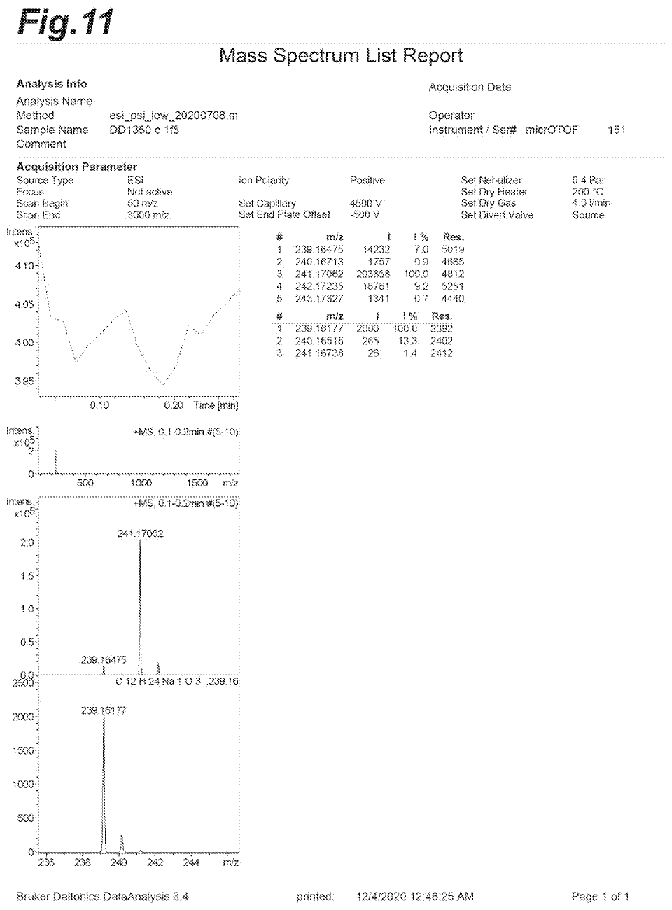

Reaction and purification were carried out in the same manner as in Example 2-2, except that the ruthenium complex of Formula (V) obtained in Example 1-2 was used as a catalyst instead of the ruthenium complex of Formula [IV] obtained according to Example 1-1. The product obtained in the same manner as in Example 2-2 was analyzed by ¹H-NMR, and it was confirmed that the target product 7-hydroxy-3,7-dimethyloctyl acetate (28.4 mg, yield: 65%) was obtained. Analysis was performed with a time-of-flight mass spectrometer (ESI-TOF-MS), and it was confirmed that the labeling ratio (enrichment factor) with oxygen-18 isotope (¹⁸O)) was 93 atom %. FIG. 11 shows the analysis results obtained using a time-of-flight mass spectrometer.

### Example 2-4

### <Hydroxylation>

Hydroxylation of 4-methylpentyl benzoate represented by the following Formula (2-4) was carried out by using the ruthenium complex of Formula [IV] (wherein R in Formula [IV] is a 2,6-dimethylphenyl group) obtained according to Example 1-1 as a catalyst, under a nitrogen gas stream and at a reaction temperature of 35° C. Specifically, 3.18 mg (4.0 μmol) of the ruthenium complex of Formula [IV] was introduced into a 5-mL Schlenk tube, the interior of the Schlenk tube was purged with nitrogen, and then 0.5 mL of 1,1,2,2-tetrachloroethane, 41.3 mg (0.20 mmol) of 4-methylpentyl benzoate, and 8.0 mg (7.2 μL, 0.40 mmol) of ¹⁸O-labeled water (≥98 atom %) were introduced into the Schlenk tube. In addition, 250 mg (0.40 mmol) of the oxidant raw material of Formula (VI) obtained in Example 1-3 was added thereto, and the mixture was stirred at 35° C. for 24 hours.

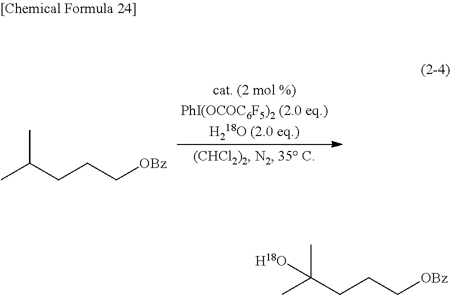

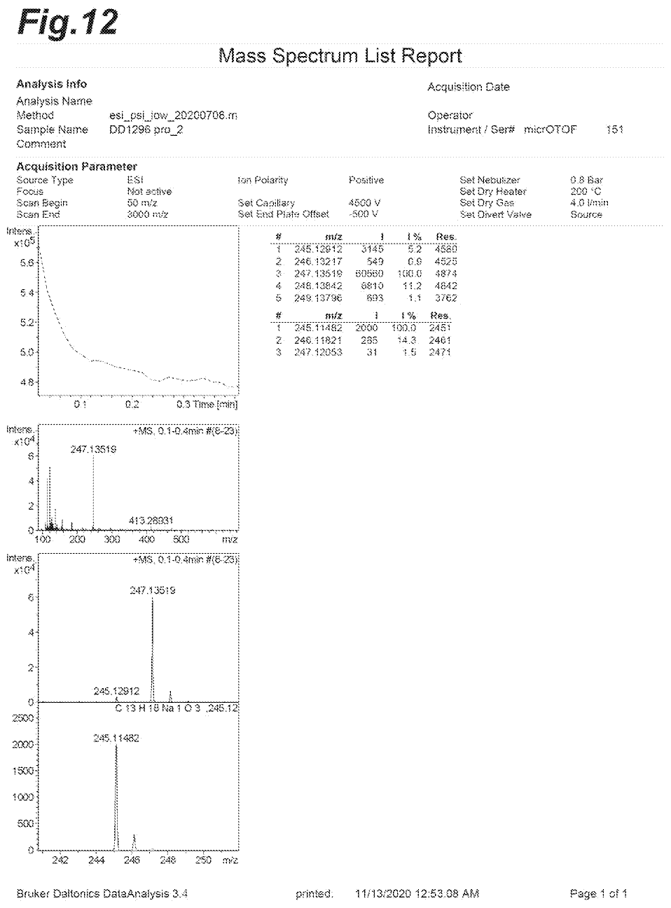

After completion of stirring, the reaction mixture was subjected to the removal of carboxylic acids and the catalyst by using short column chromatography (basic silica gel, developing solvent: ethyl acetate), and then the solvent was distilled off by using a rotary evaporator. The obtained mixture was purified by column chromatography (neutral silica gel, developing solvent: hexane/ethyl acetate=4/1 to 2/1), and then the solvent was distilled off by using a rotary evaporator. The obtained mixture was analyzed by ¹H-NMR, and it was confirmed that the target product 4-hydroxy-4-methylpentyl benzoate (34.5 mg, yield: 77%) was obtained. Analysis was performed with a time-of-flight mass spectrometer (ESI-TOF-MS), and it was confirmed that the labeling ratio (enrichment factor) with oxygen-18 isotope (¹⁸O) was 95 atom %. FIG. 12 shows the analysis results obtained using a time-of-flight mass spectrometer.

### Example 3-1

### Synthesis of 1,6-anhydro-4-O-methyl-2,3-O-isopropylidene-β-D-mannopyranose

Synthesis represented by the following Reaction Scheme (3-0) was carried out by the following method by referring to a known synthesis method (J. Org. Chem., 1989, 54, 6125-6127, J. Org. Chem. 1989, 54, 1346-1353.). First, etherification of D-mannose was carried out by the following procedure at a temperature of from 0° C. to room temperature under nitrogen, and 1,6-anhydro-β-D-mannopyranose (compound VII) was obtained. Specifically, a 25-mL dropping funnel was attached to a 100-mL Schlenk tube, 4.00 g (22.2 mmol, 1.0 equivalent) of D-mannose was introduced into the flask, while 5.50 g (28.9 mmol, 1.3 equivalents) of p-toluenesulfonyl chloride was introduced into the dropping funnel, and nitrogen purging was performed. 40 mL of pyridine was introduced into the flask, while 8 mL of pyridine was introduced into the dropping funnel, to dissolve the compounds, respectively, and then the flask was immersed in an ice bath to be cooled to 0° C.

Thereafter, while the reaction solution in the flask was stirred at 0° C., a solution of sulfonyl chloride was added dropwise thereto over 10 minutes. The mixture was stirred at room temperature for 2 hours and then cooled again to 0° C., and 12 mL (60 mmol, 2.7 equivalents) of a 5 Normal aqueous solution of sodium hydroxide was added dropwise from the dropping funnel over 10 minutes. The mixture was stirred at room temperature for 2 hours and then cooled again to 0° C., and the mixture was neutralized to pH 7.0 with 2 Normal hydrochloric acid. The obtained reaction mixture was concentrated under reduced pressure by using a rotary evaporator, and the solvent was distilled off. In addition, an operation of adding 20 mL of toluene and concentrating the mixture under reduced pressure by using a rotary evaporator was repeatedly carried out two times.

The mixture dried under reduced pressure was suspended in 100 mL of ethanol and filtered, and the remaining solid was washed three times with 30 mL of ethanol. The solvent was distilled off from the obtained filtrate by using a rotary evaporator and was dried under reduced pressure. The mixture thus obtained included 1,6-anhydro-β-D-mannopyranose represented by Formula (VII) in the following Reaction Scheme (3-0). This mixture was used in the subsequent reaction without being purified.

Next, isopropylidene protection of the above-described 1,6-anhydro-β-D-mannopyranose was performed by the following procedure at room temperature under nitrogen, and 1,6-anhydro-2,3-O-isopropylidene-β-D-mannopyranose represented by Formula (VIII) in the following Reaction Scheme (3-0) was obtained. Specifically, the mixture obtained by the above-described reaction was introduced into a 300-mL eggplant-shaped flask, the flask was purged with nitrogen, subsequently 70 mL of acetone was added thereto, and the mixture was suspended by stirring. Subsequently, 6.94 g (8.19 mL, 66.6 mmol, 3.0 equivalents) of 2,2-dimethoxypropane and 211 mg (1.11 mmol, 0.05 equivalents) of p-toluenesulfonic acid monohydrate were added thereto, and the mixture was stirred at room temperature for 12 hours to obtain a reaction mixture.

112 mg (155 μL, 1.11 mmol, 0.05 equivalents) of triethylamine was added to this reaction mixture to neutralize the reaction mixture, salts were removed by short column chromatography (neutral silica gel, developing solvent: ethyl acetate), and then the solvent was distilled off by using a rotary evaporator. The obtained mixture was purified by column chromatography (neutral silica gel, developing solvent: hexane/ethyl acetate=1/1 to 1/2), and the solvent was distilled off by using a rotary evaporator. The obtained product was analyzed by ¹H-NMR, and it was confirmed that 2.32 g (11.5 mmol) of 1,6-anhydro-2,3-O-isopropylidene-β-D-mannopyranose (the following Formula (VIII), two-step yield: 51.8%) was obtained.

Methyl etherification of the above-mentioned 1,6-anhydro-2,3-O-isopropylidene-β-D mannopyranose was carried out by the following procedure at a temperature of from 0° C. to room temperature under nitrogen, and 1,6-anhydro-4-O-methyl-2,3-O-isopropylidene-β-D-mannopyranose of the following Formula (IX) was obtained. Specifically, 1.01 g (5.00 mmol, 1.0 equivalent) of 1,6-anhydro-2,3-O-isopropylidene-β-D-mannopyranose of Formula (VIII) was introduced into a 100-mL Schlenk flask, and the flask was purged with nitrogen. Thereafter, 20 mL of tetrahydrofuran and 5 mL of N,N-dimethylformamide were added thereto, and the mixture was cooled to 0° C. Subsequently, 300 mg (7.50 mmol, 1.5 equivalents) of sodium hydride (60%) was added thereto, and the mixture was stirred at 0° C. for 1 hour. Thereafter, 1.42 g (625 μL, 10.0 mmol, 2.0 equivalents) of iodomethane was added dropwise thereto for 5 minutes at 0° C.

The mixture was stirred at room temperature for 1.5 hours, subsequently 20 mL of a saturated aqueous solution of ammonium chloride was added thereto at 0° C., and an extraction operation was carried out three times with 20 mL of ethyl acetate. The organic phases of three batches were combined, washed with 20 mL of saturated brine, and dried over sodium sulfate. Thereafter, the solvent was distilled off by using a rotary evaporator to obtain a mixture. The obtained mixture was purified by using column chromatography (neutral silica gel, developing solvent: hexane/ethyl acetate=9/1 to 2/1), and the solvent was distilled off by using a rotary evaporator. The obtained product was analyzed by ¹H-NMR, and it was confirmed that 1.02 g (4.72 mmol) of 1,6-anhydro-4-O-methyl-2,3-O-isopropylidene-β-D-mannopyranose (Formula (IX) in the following Reaction Scheme (3-0), yield: 94.4%) was obtained.

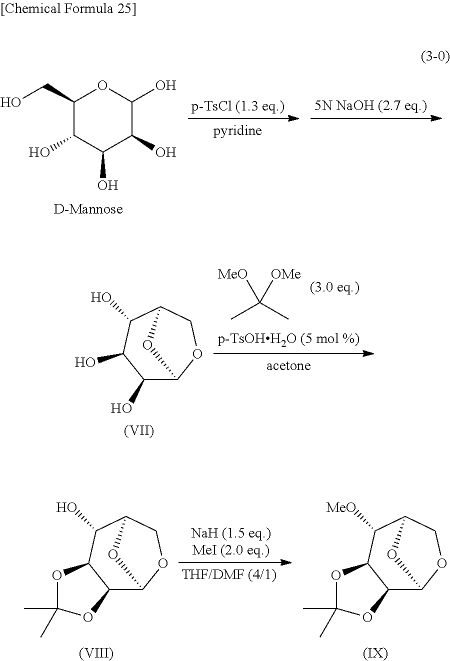

### <Oxonation>

Oxidative deprotection of 1,6-anhydro-4-O-methyl-2,3-O-isopropylidene-β-D-mannopyranose was carried out according to the following Formula (3-1) by using the ruthenium complex of Formula [IV] (wherein R in Formula [IV] is a 2,6-dimethylphenyl group) obtained according to Example 1-1 as a catalyst, under a nitrogen gas stream at a reaction temperature of 35° C. Specifically, 43.2 (0.20 mg mmol) of 1,6-anhydro-4-O-methyl-2,3-O-isopropylidene-β-D-mannopyranose and 3.18 mg (4.0 μmol) of the ruthenium complex of Formula [IV] were introduced into a 5-mL Schlenk tube, the Schlenk tube was purged with nitrogen, and then 0.5 mL of 1,1,2,2-tetrachloroethane and 8.0 mg (7.2 μL, 0.40 mmol) of ¹⁸O-labeled water (≥98 atom %) were added thereto. In addition, 250 mg (0.40 mmol) of the oxidant raw material of Formula (VI) obtained in Example 1-3 was added thereto, and the mixture was stirred at 35° C. for 3 hours.

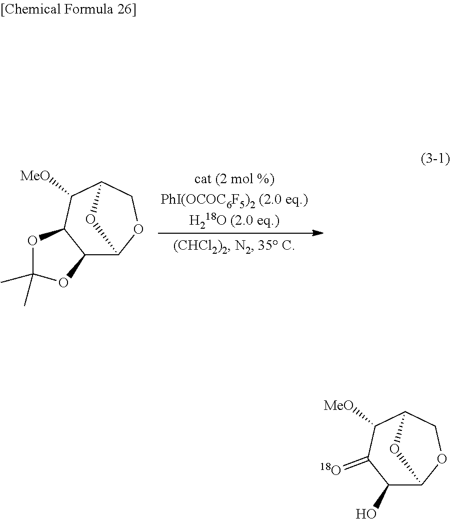

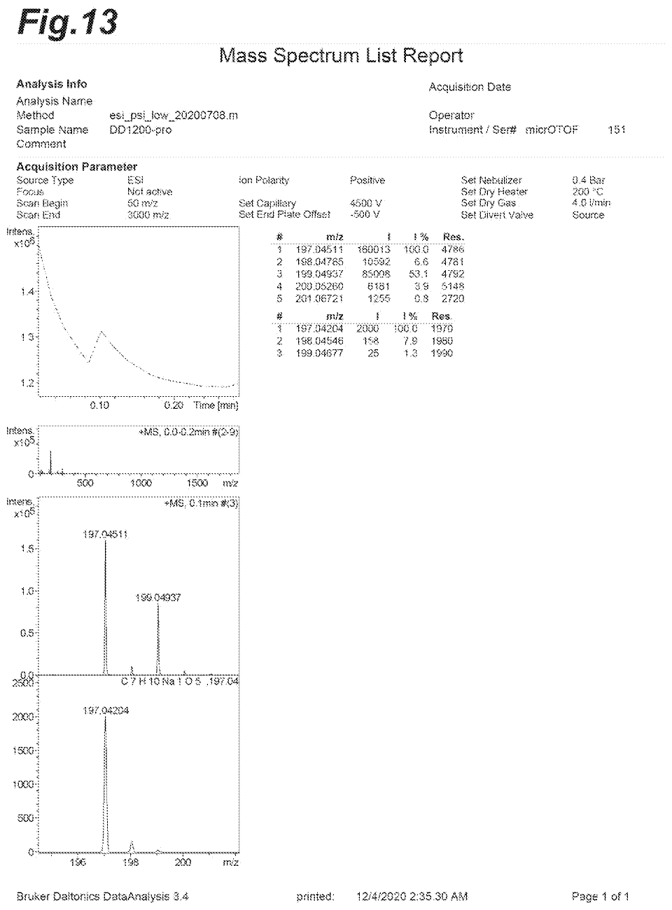

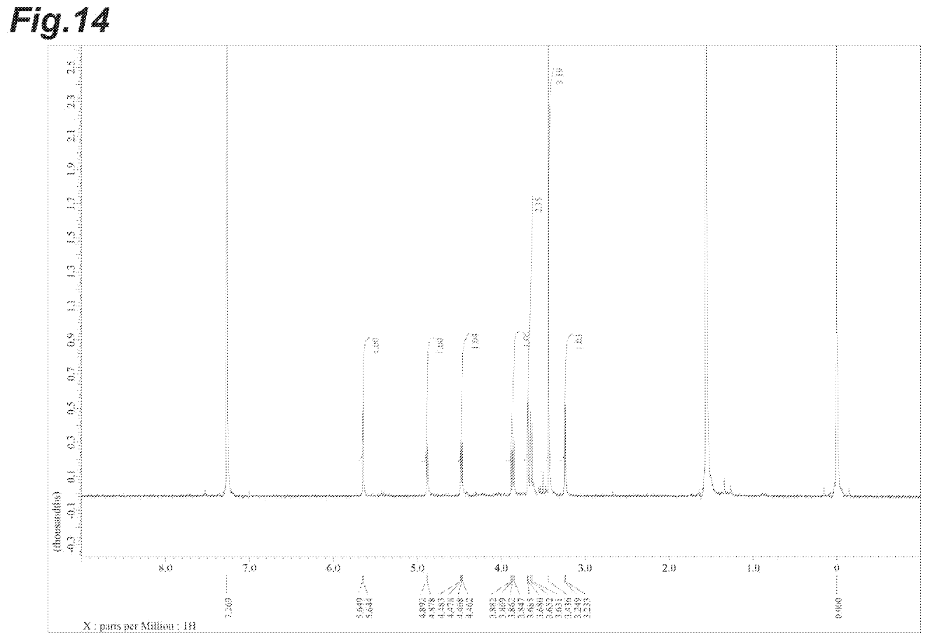

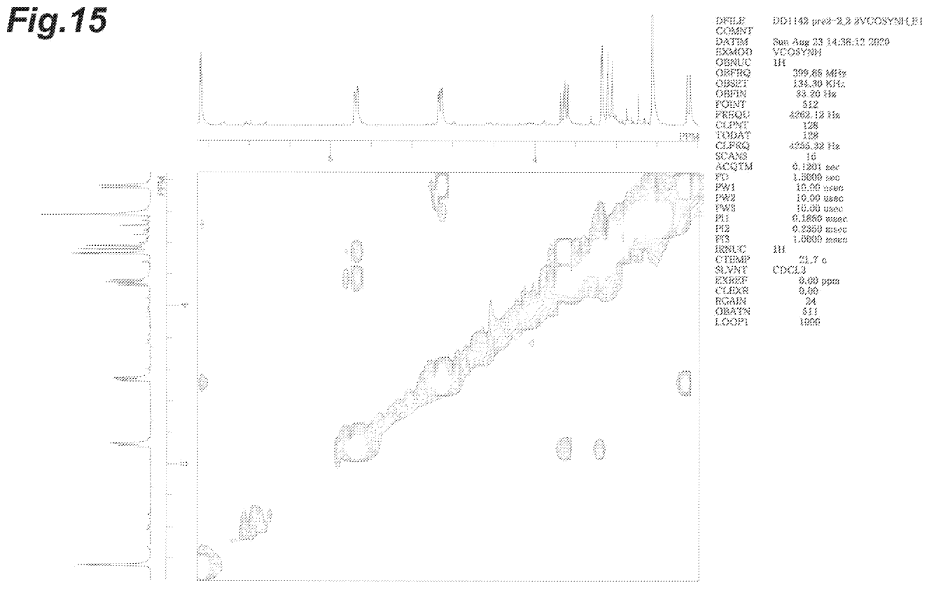

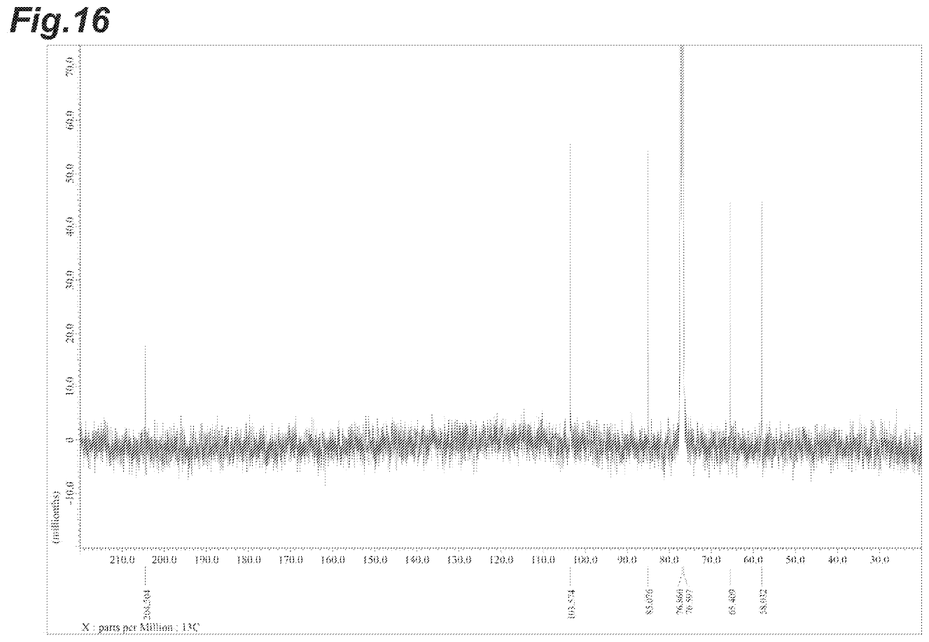

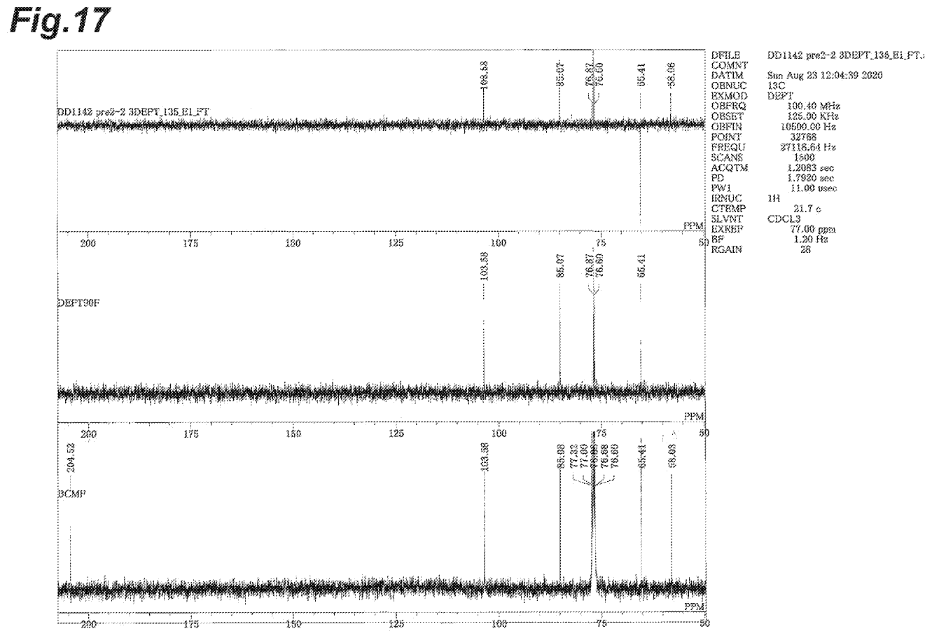

After completion of stirring, the reaction mixture was purified by using column chromatography (neutral silica gel, developing solvent: hexane/ethyl acetate=4/1 to 1/1). Thereafter, the solvent was distilled off by using a rotary evaporator. The obtained product was analyzed by ¹H-NMR, and it was confirmed that the target product (1R,2R,4R,5R)-4-hydroxy-2-methoxy-6,8-dioxabicyclo[3.2.1]octan-3-one (11.3 mg, yield: 32%) was obtained. Analysis was performed with a time-of-flight mass spectrometer (ESI-TOF-MS), and it was confirmed that the labeling ratio (enrichment factor) with oxygen-18 isotope (¹⁸O) was 34 atom %. FIG. 13 shows the analysis results obtained using a time-of-flight mass spectrometer. FIG. 14 shows the results of ¹H-NMR analysis, and FIG. 15 shows the results of two-dimensional NMR analysis. FIG. 16 shows the results of ¹³C-NMR analysis based on BCM, and FIG. 17 shows the results of ¹³C-NMR analysis based on a DEPT method. The product (1R,2R,4R,5R)-4-hydroxy-2-methoxy-6,8-dioxabicyclo[3.2.1]octan-3-one showed a tendency that the labeling ratio with oxygen-18 isotope (¹⁸O) decreases over time. For this reason, the following one-pot synthesis was carried out.

### Example 3-2

### <Oxonation→Hydroxylation>

One-pot synthesis represented by the following Formula (3-2) was carried out. Oxidative deprotection of 1,6-anhydro-4-O-methyl-2,3-O-isopropylidene-β-D-mannopyranose was carried out according to the following Formula (3-2) by using the ruthenium complex of Formula [IV] (wherein R in Formula [IV] is a 2,6-dimethylphenyl group) obtained according to Example 1-1 as a catalyst, under a nitrogen gas stream at a reaction temperature of 35° C. Specifically, 43.2 mg (0.20 mmol) of 1,6-anhydro-4-O-methyl-2,3-O-isopropylidene-β-D-mannopyranose and 3.18 mg (4.0 μmol) of the ruthenium complex of Formula [IV] were introduced into a 5-mL Schlenk tube, the Schlenk tube was purged with nitrogen, and then 0.5 mL of 1,1,2,2-tetrachloroethane and 8.0 mg (7.2 μL, 0.40 mmol) of ¹⁸O-labeled water (≥98 atom %) were added thereto. In addition, 250 mg (0.40 mmol) of the oxidant raw material of Formula (VI) obtained in Example 1-3 was added thereto, and the mixture was stirred at 35° C. for 3 hours.

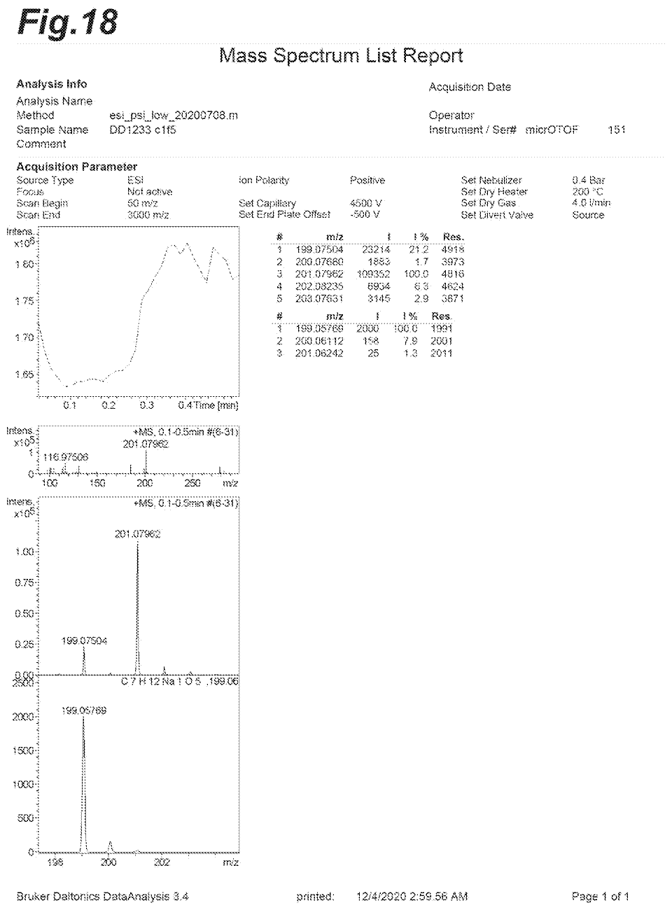

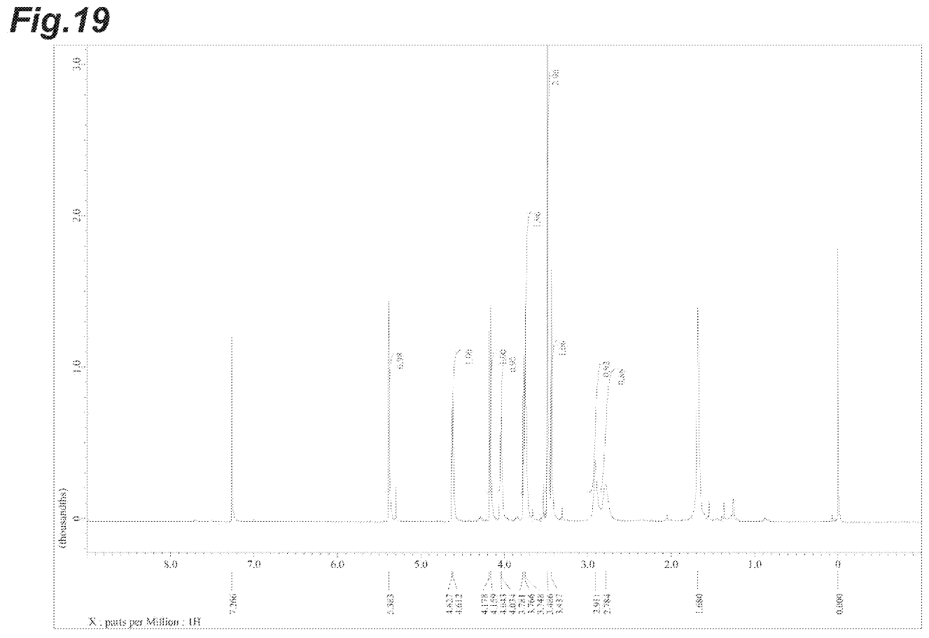

Subsequently, 0.5 mL of methanol was added thereto, the mixture was cooled to 0° C., subsequently sodium cyanoborohydride (44.0 mg, 0.70 mmol) was added thereto, and the mixture was stirred at 0° C. for 10 minutes to obtain a reaction mixture. After completion of stirring, carboxylic acids, the catalyst, and the reducing agent were removed from the reaction mixture by using short column chromatography (basic silica gel, developing solvent: dichloromethane/methanol=9/1), and then the solvent was distilled off by using a rotary evaporator. The obtained mixture was purified by column chromatography (neutral silica gel, developing solvent: dichloromethane/methanol=9/1 to 4/1), and the solvent was distilled off by using a rotary evaporator. The obtained product was analyzed by ¹H-NMR, and it was confirmed that 1,6-anhydro-4-O-methyl-β-D-mannopyranose (4.1 mg, yield: 12%) was obtained. Analysis was performed with a time-of-flight mass spectrometer (ESI-TOF-MS), and the labeling ratio (enrichment factor) of 1,6-anhydro-4-O-methyl-β-D-mannopyranose with oxygen-18 isotope (¹⁸O) was 82 atom %. FIG. 18 shows the results of analysis using a time-of-flight mass spectrometer. FIG. 19 shows the results of ¹H-NMR analysis.

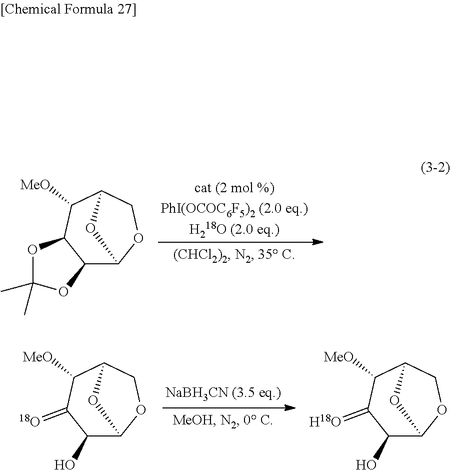

### Example 3-3

<Synthesis of Mannose Labeled with O¹⁸>

Synthesis represented by the following Formula (3-3) was carried out. 17.6 mg (0.10 mmol) of 1,6-anhydro-4-O-methyl-β-D-mannopyranose was introduced into a 5-mL Schlenk tube, and the interior of the Schlenk tube was purged with nitrogen. Thereafter, 1 mL of dichloromethane was added thereto, and the mixture was cooled to 0° C. 0.30 mL (0.30 mmol) of boron tribromide (1 M dichloromethane solution) was added thereto, and the mixture was stirred at 0° C. for 30 minutes to obtain a reaction mixture.

1 mL of water was added to the reaction mixture, and 79.1 mg (1.0 mmol) of pyridine was added thereto to neutralize the mixture. The solvent was distilled off by using a rotary evaporator. The obtained mixture was analyzed by ¹H-NMR, and it was confirmed that mannose was obtained as a main product.

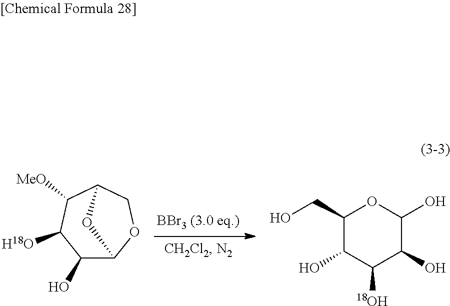 [SMILES: CO[C@H]1[C@H]([18OH])[C@H](O)[C@@H]2OC[C@H]1O2 ; OC[C@H]1OC(O)[C@@H](O)[C@@H]([18OH])[C@@H]1O]

### Example 4-1

A trans-form ruthenium complex and a cis-form ruthenium complex, in which R in the following General Formulas (10) and (11) was H (hydrogen), were obtained. These could be synthesized by treating the compound of Formula (I) in Reaction Scheme (1a) according to Example 1-1 with aqueous ammonia.

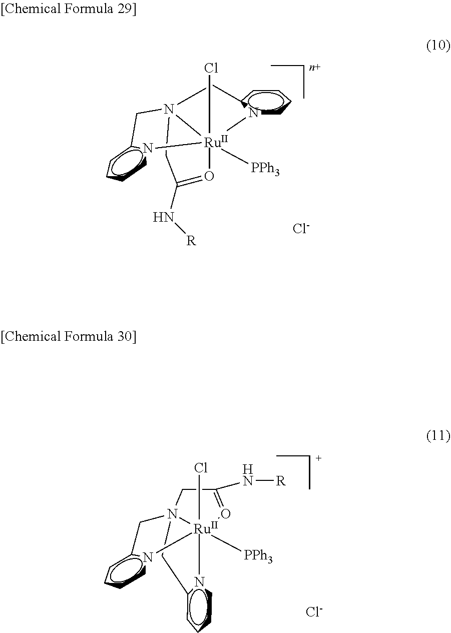

### Examples 4-2 to 4-8

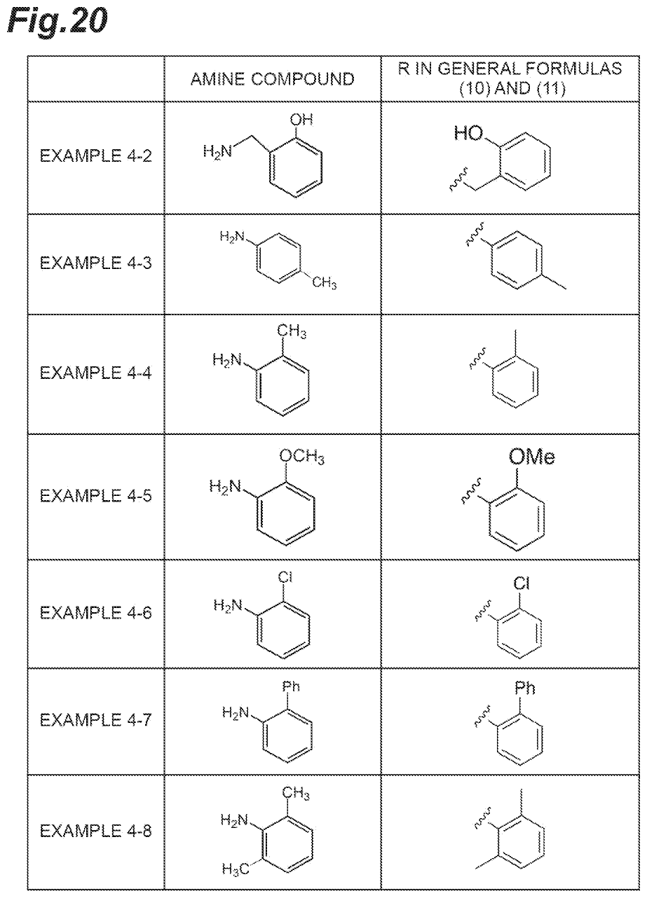

A trans-form ruthenium complex and a cis-form ruthenium complex, in which R in the above-described General Formulas (10) and (11) was a substituent shown in FIG. 20, were respectively obtained. These were each synthesized by using the amine compound shown in FIG. 20 instead of the aniline of Formula (II) in Reaction Scheme (1c) according to Example 1-1. Incidentally, the wavy line in FIG. 20 indicates the main body part of the ruthenium complex to which R in General Formulas (10) and (11) is bonded. Furthermore, Me represents a methyl group. Thus, it was confirmed that various ruthenium complexes can be synthesized.

### Example 5-1

### <Synthesis of Ligand 2>

9.55 g (55.7 mmol, 1.0 equivalent) of methyl 4-chloro-2-pyridinecarboxylate, 50 mL of methanol, 25 ml of tetrahydrofuran, and 42.7 g (223 mmol, 4.0 equivalents) of calcium chloride were introduced into an eggplant-shaped flask. This eggplant-shaped flask was immersed in an ice water bath to be cooled to 0° C. Thereafter, 4.21 g (111 mmol, 2.0 equivalents) of sodium borohydride was slowly added thereto while the suspension was stirred at 0° C. The mixture was stirred at 0° C. for 1 hour, and then 100 mL of water was added thereto. Thereafter, an operation of blending 100 mL of ethyl acetate and then performing extraction was repeated three times. The extract obtained by three extraction operations was washed with saturated brine. Sodium sulfate was added to the washing liquid to remove moisture, and then light components were distilled off with a rotary evaporator. The obtained mixture was purified by column chromatography (neutral silica gel, developing solvent: hexane/ethyl acetate=2/1 to 1/2), and 4-chloro-2-pyridinemethanol (7.73 g, 53.8 mmol, yield: 96.7%) was obtained. The reaction scheme is as shown by the following Formula (5-1).

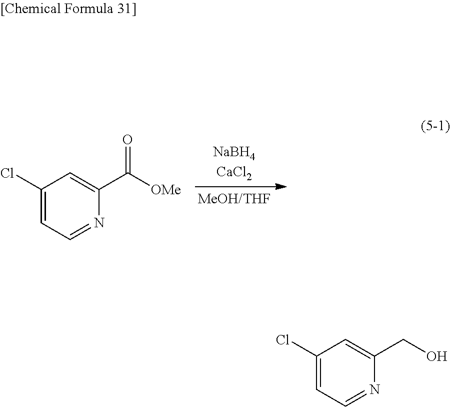 [SMILES: COC(=O)c1cc(Cl)ccn1 ; OCc1cc(Cl)ccn1]

1.44 g (10.0 mmol, 1.0 equivalent) of 4-chloro-2-pyridinemethanol and 20 mL of chloroform were introduced into an eggplant-shaped flask, and then 10.4 g (120 mmol, 12 equivalents) of manganese dioxide was added thereto. Stirring was performed for 2 hours while heating under reflux to obtain a reaction liquid. This reaction liquid was cooled to room temperature, subsequently Celite filtration was performed to remove solid components, and light components were distilled off with a rotary evaporator. The obtained mixture was purified by column chromatography (neutral silica gel, developing solvent: dichloromethane/methanol=1/0 to 9/1), and 4-chloro-2-pyridinecarboxaldehyde (947 mg, 6.69 mmol, yield: 66.9%) was obtained. The reaction scheme is as shown by the following Formula (5-2).

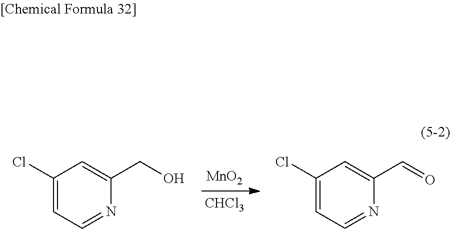

1.72 g (12.0 mmol, 1.0 equivalent) of 4-chloro-2-pyridinemethanol was introduced into an eggplant-shaped flask, and the flask was purged with nitrogen. Thereafter, 60 mL of dichloromethane was added thereto, and the eggplant-shaped flask was immersed in an ice water bath to be cooled to 0° C. Thereafter, 2.14 g (1.31 mL, 18.0 mmol, 1.5 equivalents) of thionyl chloride at 0° C. was added dropwise thereto for 3 minutes. After the dropwise addition, the ice water bath was removed, and stirring was performed for 2 hours while returning the temperature to room temperature. The mixture was cooled again to 0° C., and 50 mL of a saturated aqueous solution of sodium hydrogen carbonate was added thereto. Thereafter, an operation of blending 50 mL of dichloromethane and then performing extraction was repeated three times. The extract obtained by three extraction operations was washed with saturated brine. Sodium sulfate was added to the washing liquid to remove moisture, and then light components were distilled off with a rotary evaporator. The obtained mixture was purified by column chromatography (neutral silica gel, developing solvent: hexane/ethyl acetate=4/1), and 4-chloro-2-(chloromethyl)pyridine (1.71 g, 10.5 mmol, yield: 87.8%) was obtained. The reaction scheme is as shown by the following Formula (5-3).

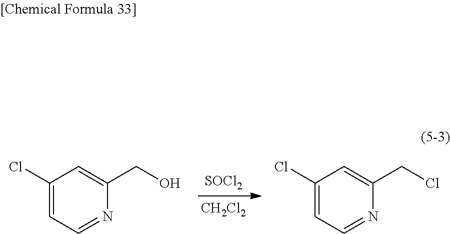

1.71 g (10.5 mmol, 1.0 equivalent) of 4-chloro-2-(chloromethyl)pyridine was introduced into an eggplant-shaped flask, the flask was purged with nitrogen, and then 21 mL of N,N-dimethylformamide and 2.15 g (11.6 mmol, 1.1 equivalents) of potassium phthalimide were added thereto. This was stirred at 100° C. for 2 hours and then cooled to room temperature, and 50 mL of a saturated aqueous solution of sodium hydrogen carbonate was added thereto. Thereafter, an operation of blending 50 mL of dichloromethane and then performing extraction was repeated three times. The extract obtained by three extraction operations was washed with saturated brine. Sodium sulfate was added to the washing liquid to remove moisture, and then light components were distilled off with a rotary evaporator. The mixture thus obtained included 2-((4-chloro-2-pyridyl)methyl) isoindoline-1,3-dione. The reaction scheme is as shown by the following Formula (5-4). This mixture was used for the subsequent reaction without being purified.

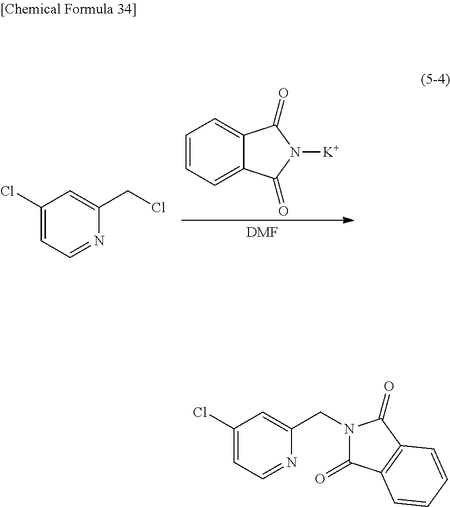

The mixture obtained by the above-described reaction was introduced into an eggplant-shaped flask, 100 mL of ethanol and 1.58 g (1.54 mL, 31.6 mmol, 3.0 equivalents) of hydrazine monohydrate were added thereto, and stirring was performed for 2 hours while heating under reflux to obtain the reaction liquid. This reaction liquid was cooled to room temperature, subsequently filtration was performed to remove solid components, and light components were distilled off with a rotary evaporator. The obtained mixture was purified by column chromatography (basic silica gel, developing solvent: dichloromethane/methanol=1/0 to 99/1), and 4-chloro-2-pyridinemethanamine (1.22 g, 8.53 mmol, two-step yield: 80.9%) was obtained. The reaction scheme is as shown by the following Formula (5-5).

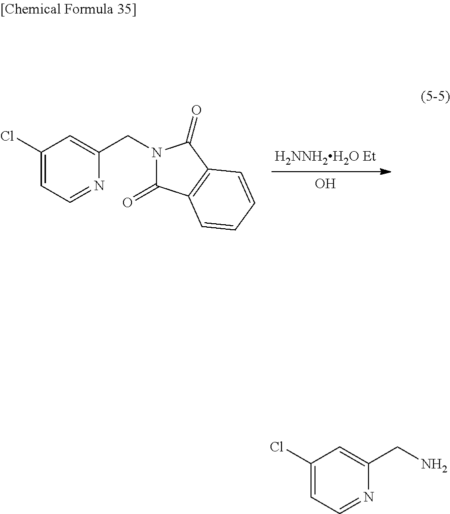 [SMILES: O=C1c2ccccc2C(=O)N1Cc1cc(Cl)ccn1 ; O ; NCc1cc(Cl)ccn1]

713 mg (5.00 mmol, 1.0 equivalent) of 4-chloro-2-pyridinemethanamine, 10 mL of 1,2-dichloroethane, and 708 mg (5.00 mmol, 1.0 equivalent) of 4-chloro-2-pyridinecarboxaldehyde were introduced into an eggplant-shaped flask. This eggplant-shaped flask was immersed in an ice water bath to be cooled to 0° C. Thereafter, while stirring the reaction solution at 0° C., 2.12 g (10.0 mmol, 2.0 equivalents) of sodium triacetoxyborohydride was slowly added thereto, the ice water bath was removed, and stirring was performed for 3 hours while returning the temperature to room temperature. The reaction mixture was cooled again to 0° C., 20 mL of a saturated aqueous solution of sodium hydrogen carbonate was added thereto, and then an operation of blending 20 mL of dichloromethane and performing extraction was repeated three times. The obtained extract was washed with saturated brine, sodium sulfate was added to the washing liquid to remove moisture, and light components were distilled off with a rotary evaporator. The obtained mixture was purified by column chromatography (basic silica gel, developing solvent: hexane/ethyl acetate 4/1 to 2/1), and bis((4-chloro-2-pyridyl)methyl)amine (1.16 g, 4.34 mmol, yield: 86.8%) was obtained. The reaction scheme is as shown by the following Formula (5-6).

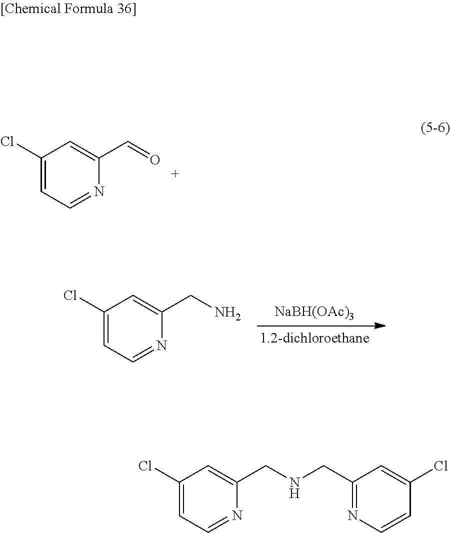 [SMILES: O=Cc1cc(Cl)ccn1 ; NCc1cc(Cl)ccn1 ; Clc1ccnc(CNCc2cc(Cl)ccn2)c1 ; "NaBH(OAc)3"]

An eggplant-shaped flask was purged with nitrogen, subsequently 2.42 g (2.47 mL, 20.0 mmol, 1.0 equivalent) of 2,6-dimethylaniline, 40 mL of dichloromethane, and 2.23 g (3.07 mL, 22.0 mmol, 1.1 equivalents) of triethylamine were introduced into the flask, and the flask was immersed in an ice water bath to be cooled to 0° C. Thereafter, 2.48 g (1.75 mL, 22.0 mmol, 1.1 equivalents) of chloroacetyl chloride was added dropwise thereto for 3 minutes at 0° C., the ice water bath was removed, and stirring was performed for 1 hour while returning the temperature to room temperature. The mixture was cooled again to 0° C., 40 mL of a saturated aqueous solution of sodium hydrogen carbonate was added thereto, and then an operation of blending 40 mL of dichloromethane and performing extraction was repeated three times. The extract obtained by three extraction operations was washed with 1 Normal hydrochloric acid and saturated brine, sodium sulfate was added to the washing liquid to remove moisture, and then light components were distilled off with a rotary evaporator. The obtained mixture was purified by column chromatography (neutral silica gel, developing solvent: dichloromethane/methanol=1/0 to 49/1), and 2-chloro-N-(2,6-dimethylphenyl) acetamide (3.40 g, 17.2 mmol, yield: 86.0%) was obtained. The reaction scheme is as shown by the following Formula (5-7).

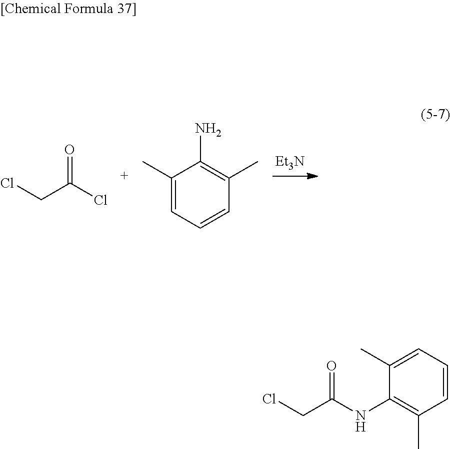

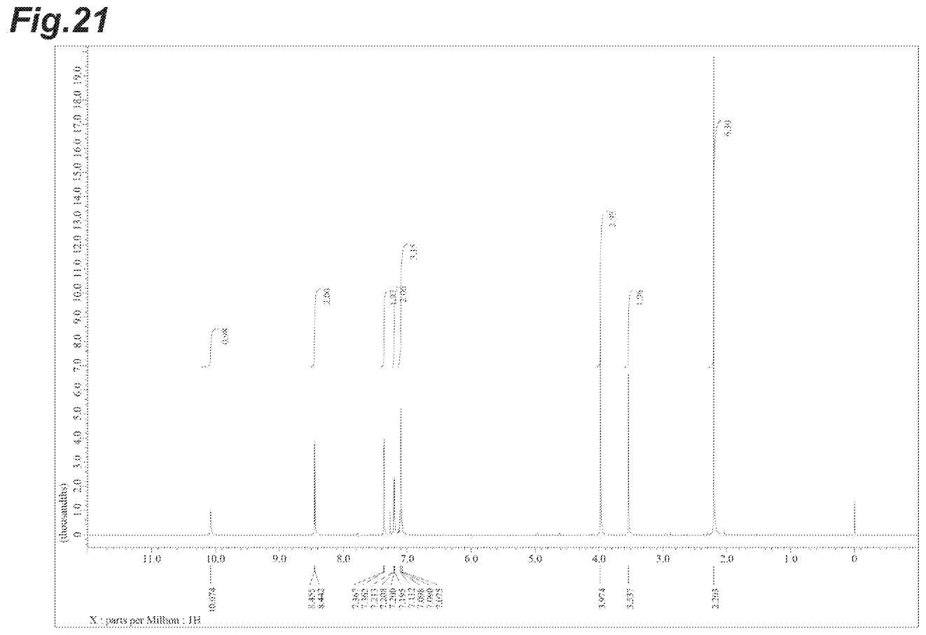

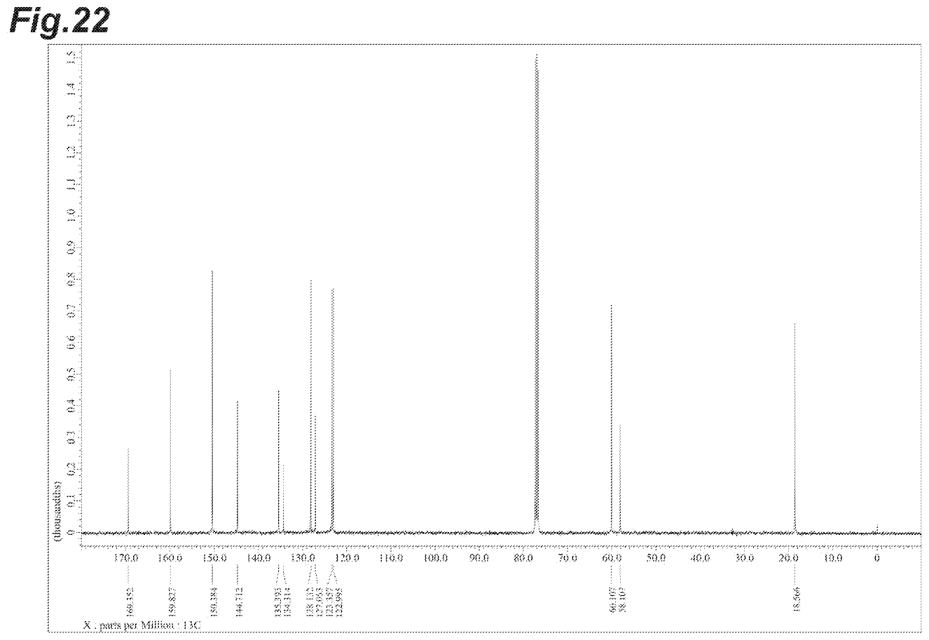

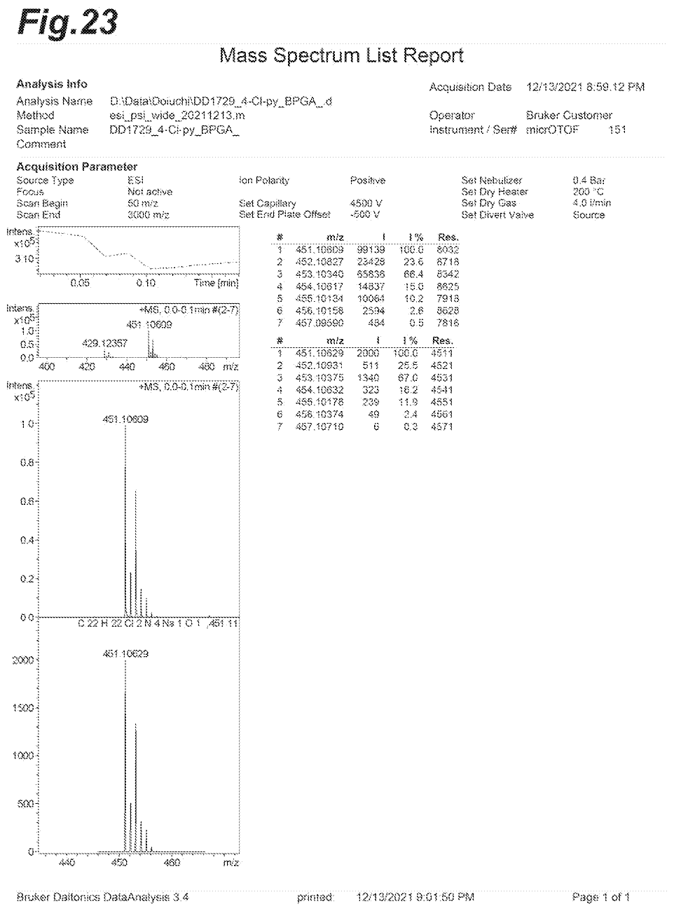

804 mg (3.00 of mmol, 1.0 equivalent) of bis((4-chloro-2-pyridyl)methyl)amine, 27 mL of acetonitrile, and 3 mL of N,N-dimethylformamide were introduced into an eggplant-shaped flask. To this eggplant-shaped flask, 771 mg (3.90 mmol, 1.3 equivalents) of 2-chloro-N-(2,6-dimethylphenyl) acetamide, 539 mg (3.90 mmol, 1.3 equivalents) of potassium carbonate, and 245 mg (1.50 mmol, 0.5 equivalents) of potassium iodide were introduced. Stirring was performed for 4 hours while heating under reflux, and a reaction liquid was obtained. This reaction liquid was cooled to room temperature, subsequently solid components were removed by using short column chromatography (basic silica gel, developing solvent: ethyl acetate), and light components were distilled off with a rotary evaporator to obtain a mixture. This mixture was purified by column chromatography (basic silica gel, developing solvent: hexane/ethyl acetate=4/1 to 1/2). The obtained product was analyzed by ¹H-NMR, ¹³C-NMR, and a time-of-flight mass spectrometer, and it was confirmed that 2-(bis((4-chloro-2-pyridyl)methyl)amino)-N-(2,6-dimethylphenyl) acetamide (1.04 g, 2.43 mmol, yield: 80.8%, ligand 2) was obtained. The reaction scheme is as shown by the following Formula (5-8). FIG. 21 shows the results of ¹H-NMR analysis of the ligand 2. FIG. 22 shows the results of ¹³C-NMR analysis of the ligand 2. FIG. 23 shows the results of analysis of the ligand 2 with a time-of-flight mass spectrometer.

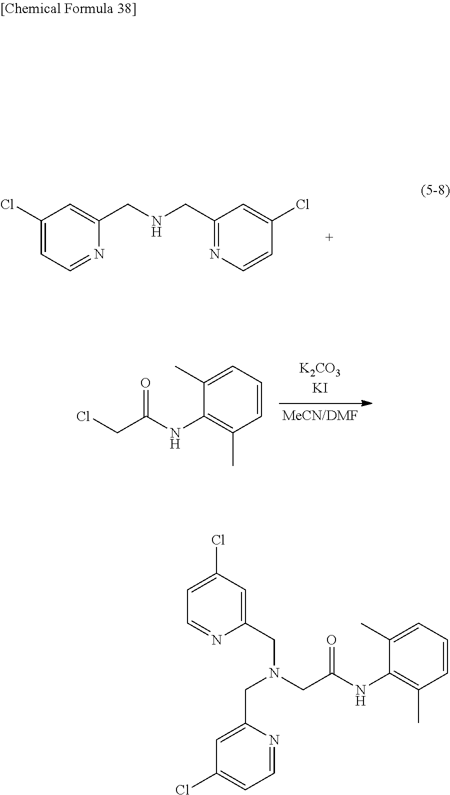 [SMILES: Clc1ccnc(CNCc2cc(Cl)ccn2)c1 ; Cc1cccc(C)c1NC(=O)CCl ; Cc1cccc(C)c1NC(=O)CN(Cc1cc(Cl)ccn1)Cc1cc(Cl)ccn1]

### <Synthesis of Metal Complex>

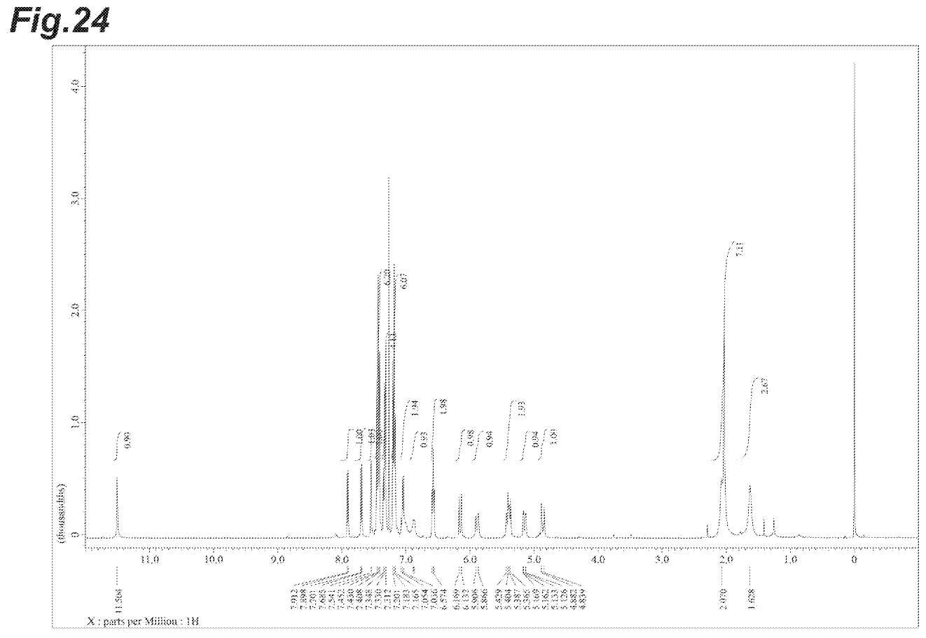

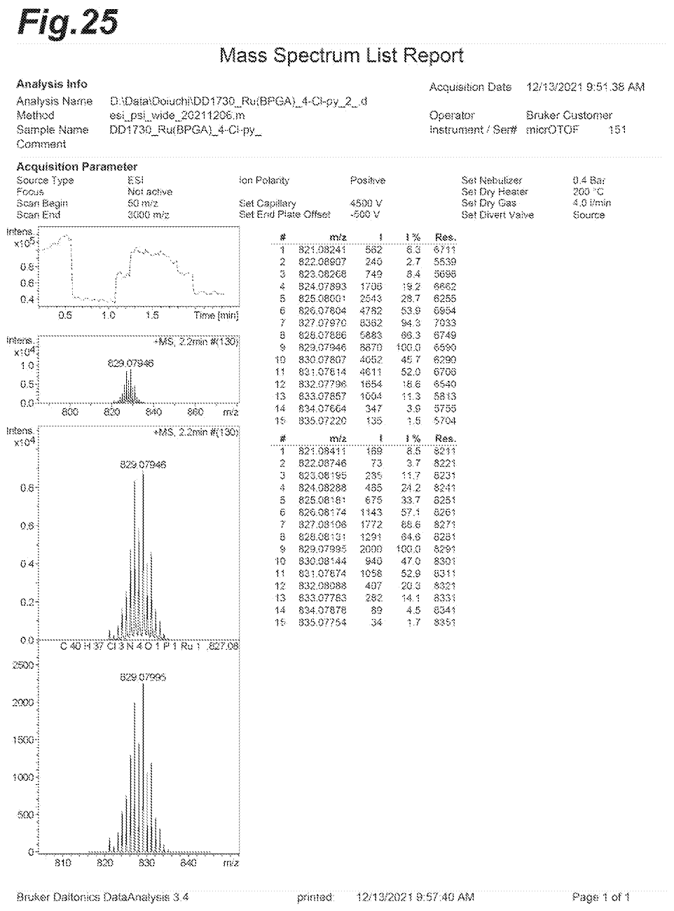

350 mg (815 μmol, 1.0 equivalent) of 2-(bis((4-chloro-2-pyridyl)methyl)amino)-N-(2,6-dimethylphenyl) acetamide (ligand 2), 860 mg (897 μmol, 1.1 equivalents) of RuCl₂(PPh₃)₃, and 32 mL of ethanol were introduced into an eggplant-shaped flask, and stirring was performed for 4 hours while heating under reflux to obtain a reaction liquid. Thereafter, light components were distilled off from the reaction liquid with a rotary evaporator. The obtained mixture was purified by two times of column chromatography (first time: neutral silica gel (developing solvent: dichloromethane/methanol=19/1 to 4/1), second time: neutral silica gel (developing solvent: chloroform/methanol=19/1 to 4/1)). The obtained product was analyzed by ¹H-NMR and a time-of-flight mass spectrometer. These analysis results were compared with the structures of the ruthenium complexes obtained in Examples 1-1 and 1-2, and it was confirmed that the product is a ruthenium complex of the following Formula (X) (168 mg, 195 μmol, yield: 23.9%). The reaction scheme is as shown by the following Formula (5-9). FIG. 24 shows the results of ¹H-NMR analysis of the ruthenium complex of Formula (X). FIG. 25 shows the results of analysis of the ruthenium complex of Formula (X) using a time-of-flight mass spectrometer.

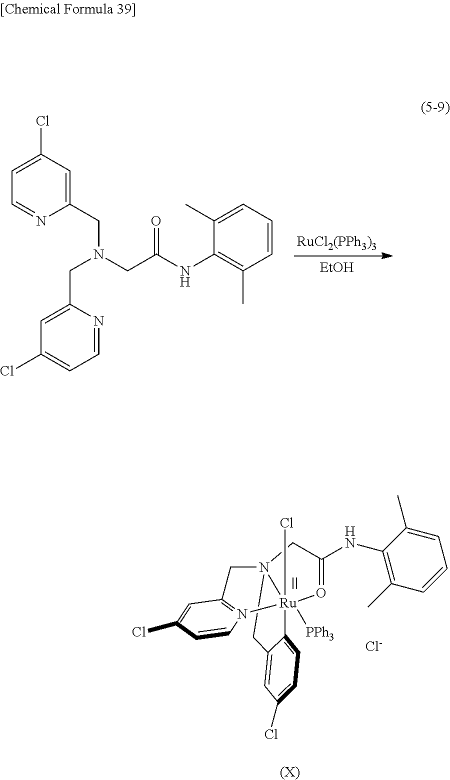

### Example 5-2

### <Synthesis of Ligand 3>

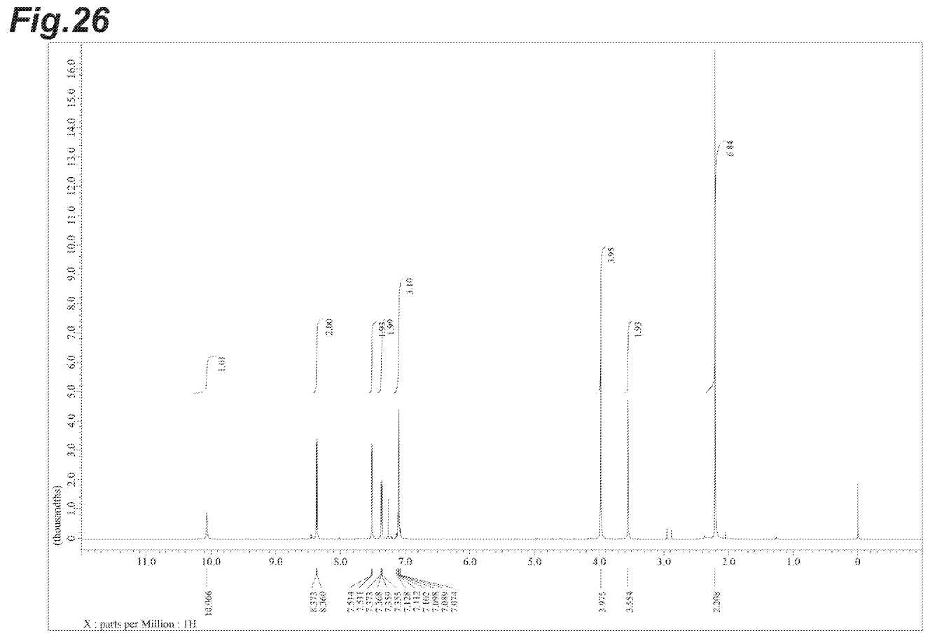

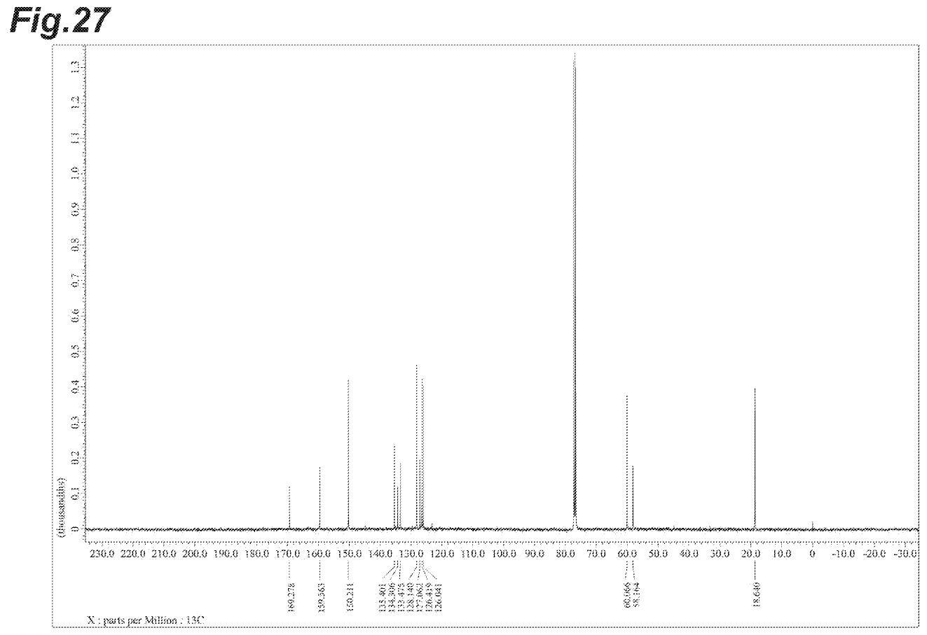

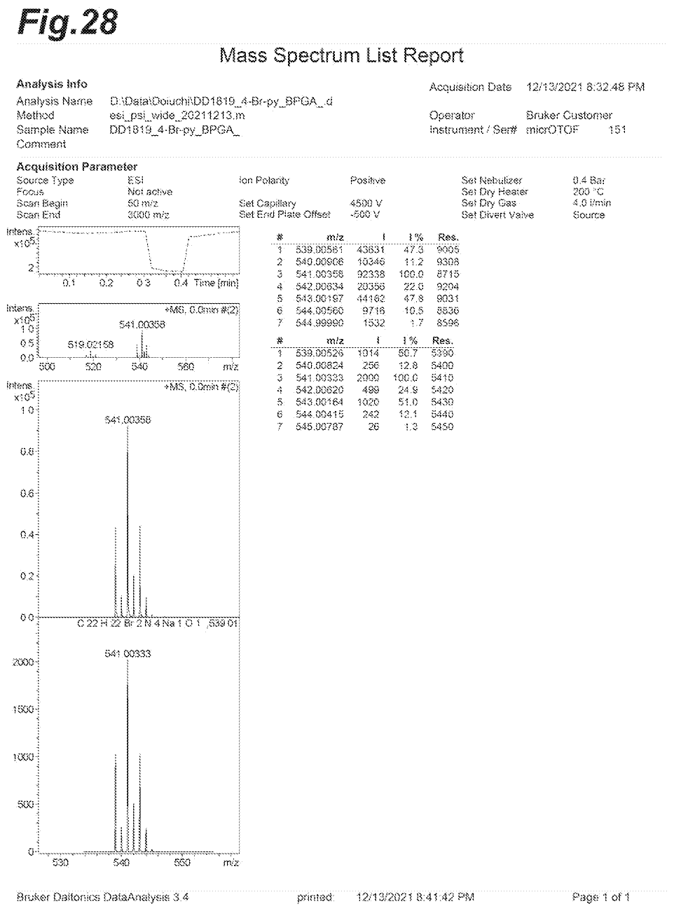

2-(Bis((4-bromo-2-pyridyl)methyl)amino)-N-(2,6-dimethylphenyl)acetamide (ligand 3) was synthesized by a reaction route represented by the following Formula (5-10) in the same manner as in Example 5-1 by using methyl 4-bromo-2-pyridinecarboxylate instead of methyl 4-chloro-2-pyridinecarboxylate. FIG. 26 shows the results of ¹H-NMR analysis of the ligand 3. FIG. 27 shows the results of ¹³C-NMR analysis of the ligand 3. FIG. 28 shows the results of analysis of the ligand 3 with a time-of-flight mass spectrometer.

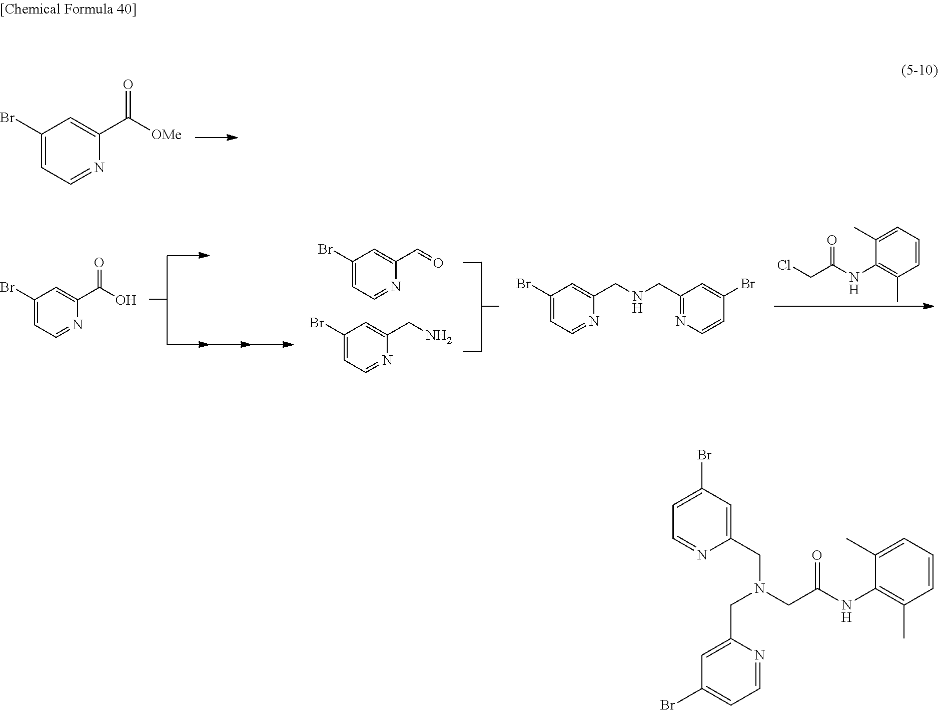

### <Synthesis of Metal Complex>

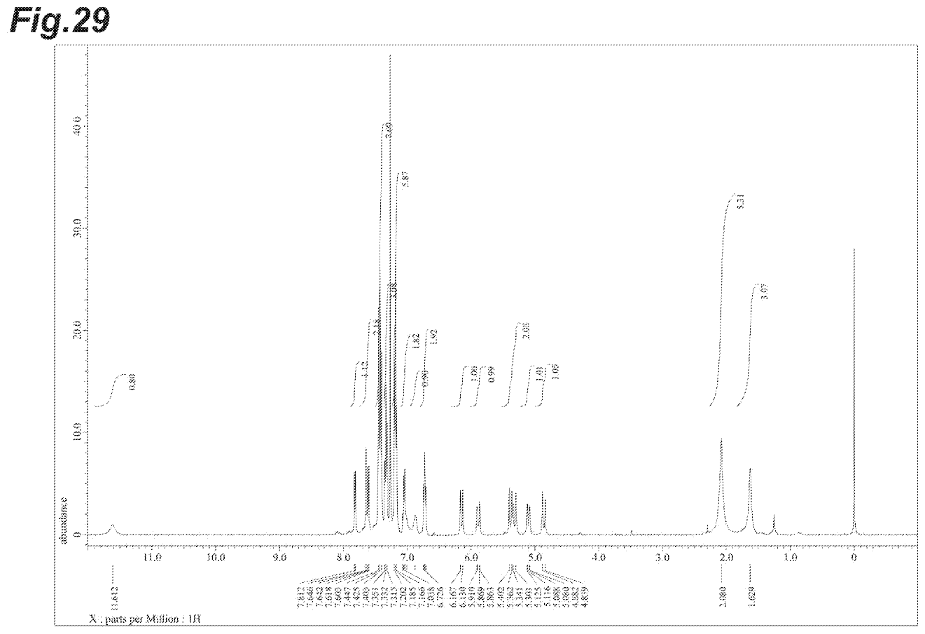

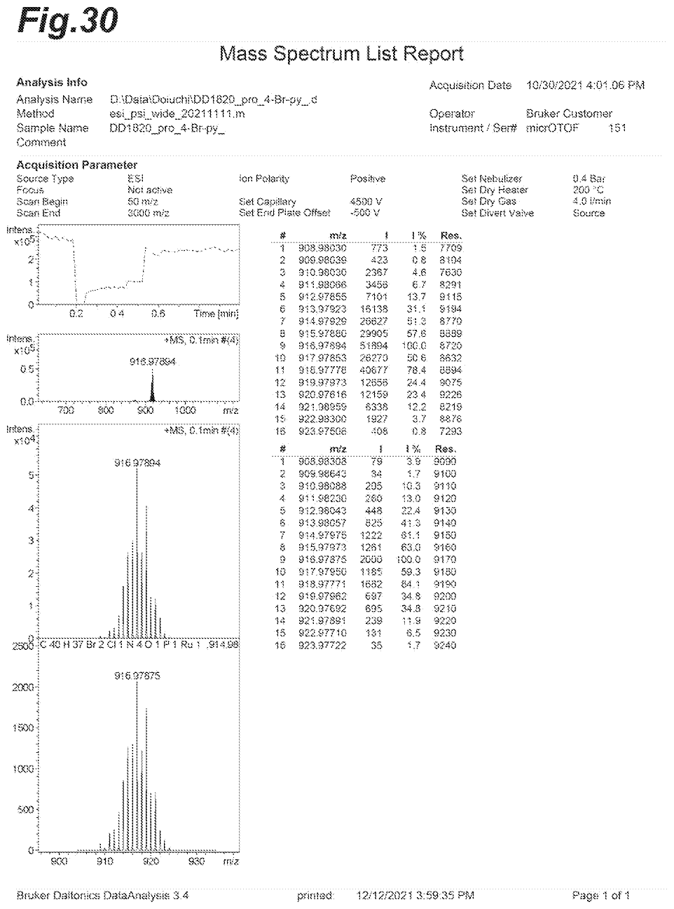

A ruthenium complex was synthesized by the same procedure as in Example 5-1, except that 2-(bis((4-bromo-2-pyridyl)methyl)amino)-N-(2,6-dimethylphenyl)acetamide (ligand 3) was used instead of 2-(bis((4-chloro-2-pyridyl)methyl)amino)-N-(2,6-dimethylphenyl)acetamide (ligand 2). The obtained product was analyzed by ¹H-NMR and a time-of-flight mass spectrometer. These analysis results were compared with the structures of the ruthenium complexes obtained in Examples 1-1 and 1-2, and it was confirmed that the product is a ruthenium complex of the following Formula (XI). The reaction scheme is as shown by the following Formula (5-11). FIG. 29 shows the results of ¹H-NMR analysis of the ruthenium complex of Formula (XI). FIG. 30 shows the results of analysis of the ruthenium complex of Formula (XI) using a time-of-flight mass spectrometer.

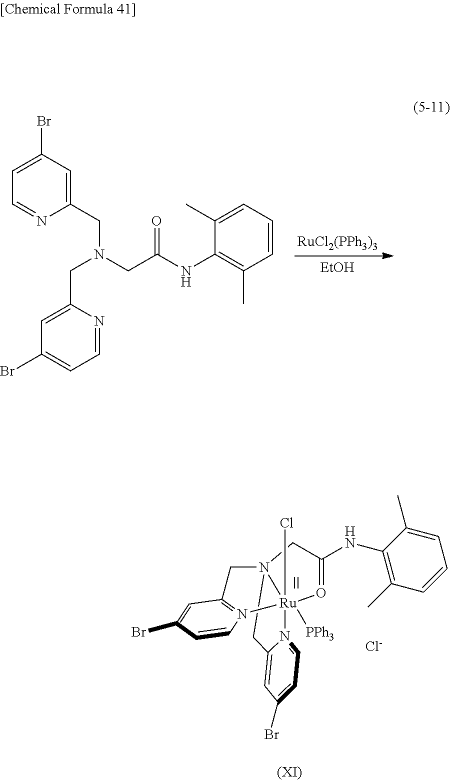

### Example 6-1

### <Hydroxylation>

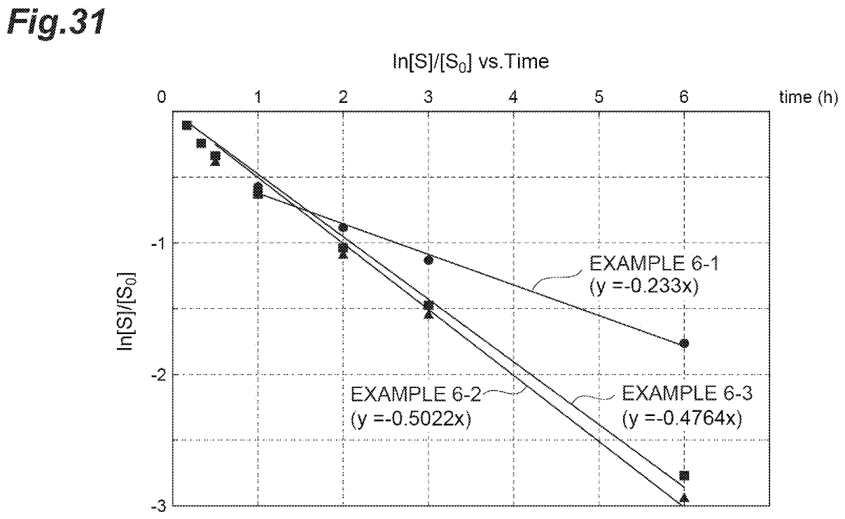

3,7-Dimethyloctyl acetate (20 mg, 0.1 mmol), iodobenzene (dipentafluorobenzoate) (123.3 mg, 0.2 mmol), and H₂O (3.6 mg, 0.2 mmol) were introduced into a 5-mL glass sample bottle. Tetrachloroethane (0.25 mL) was added to this and dissolved therein, and the mixture was adjusted to 35° C. The cis-form ruthenium complex (2.0 μmol, 2 mol %) obtained in Example 4-8 was added to this solution thus obtained, and hydroxylation as shown by the following Formula (6-1) was performed. The products produced after the passage of a predetermined time were subjected to ¹H-NMR analysis and ¹³C-NMR analysis, and the conversion ratio and the yield of each of the products were determined. The proportions of the products were traced until the reaction time reached 12 hours. The results were as shown in FIG. 31 and Table 1.

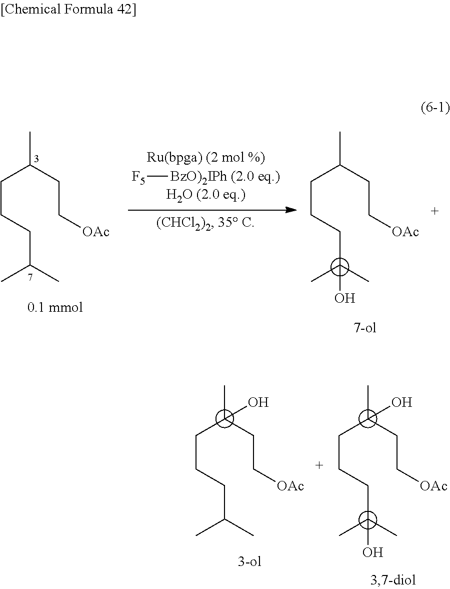

### Example 6-2

The proportions of the products were traced in the same manner as in Example 6-1, except that the above-described ruthenium catalyst of Formula (X) was used instead of the cis-form ruthenium catalyst obtained in Example 4-8. The results were as shown in FIG. 31 and Table 1.

### Example 6-3

The proportions of the products were traced in the same manner as in Example 6-1, except that the above-described ruthenium catalyst of Formula (XI) was used instead of the cis-form ruthenium catalyst obtained in Example 4-8. The results were as shown in FIG. 31 and Table 1.

TABLE 1

| Example | | Reaction time | Conversion ratio | 7-ol | 3-ol | diol | Ratio of products |
|---|---|---|---|---|---|---|---|
| No. | Catalyst | [hours] | [%] | [%] | [%] | [%] | 7-ol/3-ol/diol |
| 6-1 | Example 4-8 | 6 | 80.1 | 58.6 | 2.8 | 5.7 | 21/1/2 |
| 6-1 | Example 4-8 | 12 | 95.9 | 68.1 | 1.4 | 13.6 | 48/1/10 |
| 6-2 | Formula (X) | 6 | 94.7 | 64.8 | 1.7 | 12.7 | 39/1/8 |
| 6-3 | Formula (XI) | 6 | 93.7 | 67.3 | 1.9 | 11.5 | 35/1/6 |

The conversion ratio of the substrate and the yield of each product after the passage of 6 hours or 12 hours are shown in Table 1. The relative ratio ([S]/[S₀]) between the concentration [S] of the substrate determined from the conversion efficiency at each reaction time and the initial concentration [S₀] of the substrate was calculated. FIG. 31 is a graph showing the changes over time in the natural logarithm of the relative ratio. The gradient in this graph represents the reaction rate. It was confirmed from the results of FIG. 31 and Table 1 that the ruthenium compounds of the above-described Formula (X) and the above-described Formula (XI) have a catalytic activity two or more times that of the cis-form ruthenium compound obtained in Example 4-8.

### INDUSTRIAL APPLICABILITY

According to the present disclosure, a labeling method using an oxygen isotope, by which a labeled compound can be obtained in high yield even without using an excess of oxygen isotope-labeled water, can be provided. Furthermore, an oxygen isotope-labeled oxidant, a ruthenium complex, and a catalyst, all of which can be suitably used for such a labeling method, can be provided. Furthermore, a labeled compound labeled by means of an oxygen isotope can be provided. Furthermore, a novel compound useful as a reagent can be provided.

1. A labeling method comprising:
a step of labeling a substrate having a carbon-hydrogen bond with an oxygen isotope by using a catalyst and an oxidant produced from a hypervalent iodine compound having an ester structure and labeled water labeled with at least one oxygen isotope selected from the group consisting of ¹⁷O and ¹⁸O.

2. The labeling method according to claim 1, wherein the catalyst includes a ruthenium complex.

3. The labeling method according to claim 1, wherein the catalyst includes at least one ruthenium complex selected from the group consisting of the following General Formulas (1), (2), and (3), 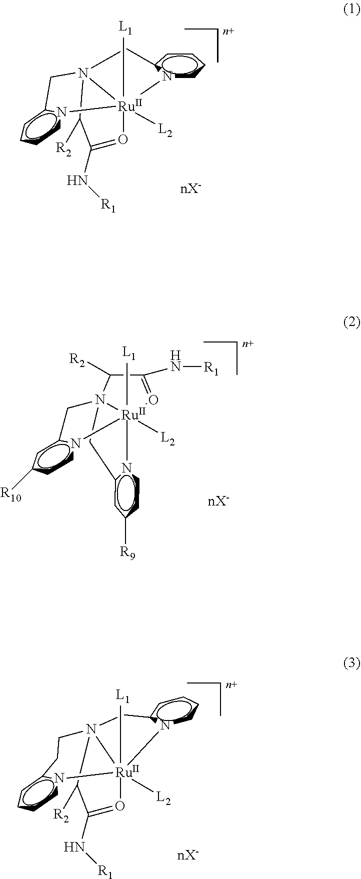
where in the General Formulas (1), (2), and (3), R₁ represents a hydrogen atom, a phenyl group, or a monovalent group in which at least one hydrogen atom of a phenyl group is substituted with an alkyl group, a hydroxy group, a phenyl group, a halogen atom, or an alkoxy group; R₂ represents a hydrogen atom, a phenyl group, or an alkyl group; L₁ represents a halogen atom or a water molecule; L₂ represents triphenylphosphine, pyridine, imidazole, or dimethyl sulfoxide; X represents a halogen atom; n represents 1 or 2; and in the General Formula (2), R₉ and R₁₀ each independently represent a hydrogen atom, a halogen atom, or an alkyl group.

4. The labeling method according to claim 1, wherein the hypervalent iodine compound includes a compound represented by the following General Formula (4), 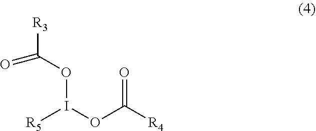
where in the General Formula (4), R₃ and R₄ each independently represent a hydrogen atom, an alkyl group, or a monovalent group having an aromatic ring; and R₅ represents a monovalent group having an aromatic ring.

5. The labeling method according to claim 1, wherein in the step, the substrate is oxidized to obtain a hydroxy compound or an oxo compound labeled with the oxygen isotope.

6. The labeling method according to claim 1, wherein in the step, an oxygen atom of a hexose included in the substrate is substituted with the oxygen isotope to label the hexose.

7. An oxidant for labeling, produced from a hypervalent iodine compound having an ester structure and labeled water labeled with at least one oxygen isotope selected from the group consisting of ¹⁷O and ¹⁸O, and labeling a substrate having a carbon-hydrogen bond with the oxygen isotope in the co-presence of a catalyst.

8. The oxidant for labeling according to claim 7, wherein the hypervalent iodine compound includes a compound represented by the following General Formula (4), 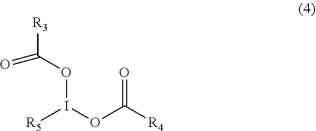
where in the General Formula (4), R₃ and R₄ each independently represent a hydrogen atom, an alkyl group, or a monovalent group having an aromatic ring; and R₅ represents a monovalent group having an aromatic ring.

9. A ruthenium complex represented by the following General Formula (2) or (3), 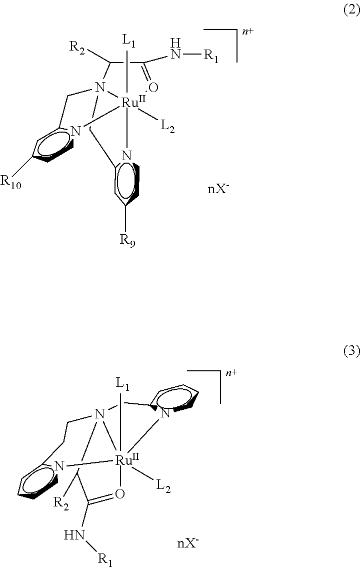
where in the General Formulas (2) and (3), R₁ represents a hydrogen atom, a phenyl group, or a monovalent group in which at least one hydrogen atom of a phenyl group is substituted with an alkyl group, a hydroxy group, a phenyl group, a halogen atom, or an alkoxy group; R₂ represents a hydrogen atom, a phenyl group, or an alkyl group; L₁ represents a halogen atom or a water molecule; L₂ represents triphenylphosphine, pyridine, imidazole, or dimethyl sulfoxide; X represents a halogen atom; n represents 1 or 2; and in the General Formula (2), R₉ and R₁₀ each independently represent a hydrogen atom, a halogen atom, or an alkyl group.

10. A catalyst comprising at least one selected from the group consisting of a ruthenium complex represented by the following General Formula (2) and a ruthenium complex represented by the following General Formula (3), 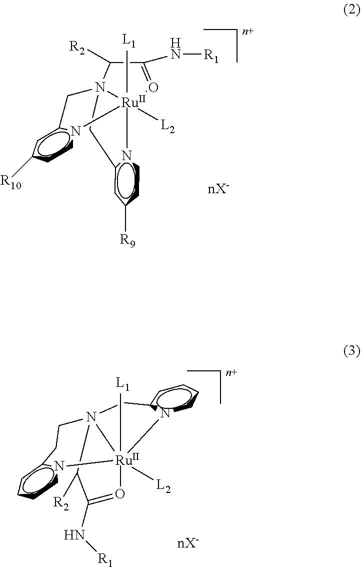
where in the General Formulas (2) and (3), R₁ represents a hydrogen atom, a phenyl group, or a monovalent group in which at least one hydrogen atom of a phenyl group is substituted with an alkyl group, a hydroxy group, a phenyl group, a halogen atom, or an alkoxy group; R₂ represents a hydrogen atom, a phenyl group, or an alkyl group; L₁ represents a halogen atom or a water molecule; L₂ represents triphenylphosphine, pyridine, imidazole, or dimethyl sulfoxide; X represents a halogen atom; n represents 1 or 2; and in the General Formula (2), R₉ and R₁₀ each independently represent a hydrogen atom, a halogen atom, or an alkyl group.

11. The catalyst according to claim 10, wherein the catalyst is an oxidation catalyst oxidizing a substrate having a carbon-hydrogen bond.

12. A labeled compound represented by the following Formula (5), (6), or (7) and labeled with at least one oxygen isotope selected from the group consisting of ¹⁷O and ¹⁸O, 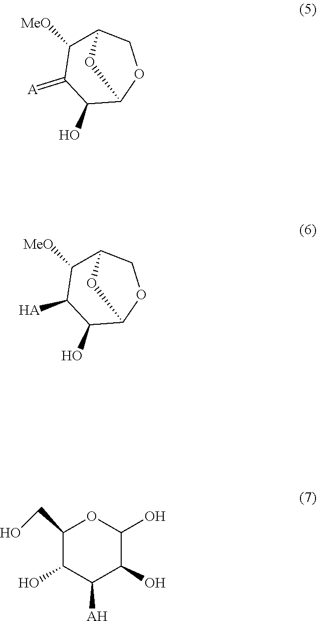
where in the Formulas (5), (6), and (7), A represents ¹⁷O or ¹⁸O.

13. A compound represented by the following Formula (8). 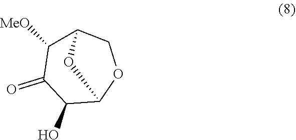 [SMILES: CO[C@H]1C(=O)[C@H](O)[C@@H]2OC[C@H]1O2]
